(19) United States
(12) Patent Application Publication
(10) Pub. No.: US 2024/0207254 A1
(43) Pub. Date: Jun. 27, 2024

(54) METHODS FOR TITRATING MITAPIVAT

(71) Applicant: Agios Pharmaceuticals, Inc., Cambridge, MA (US)

(72) Inventors: Ann Barbier, Brookline, MA (US); Christopher Bowden, Cambridge, MA (US); Peter Hawkins, Cambridge, MA (US); Varsha Venkatachalam Iyer, Boston, MA (US); Marie-Helene Jouvin, Jamaica Plain, MA (US)

(21) Appl. No.: 18/288,888

(22) PCT Filed: Apr. 28, 2022
(86) PCT No.: PCT/US2022/026833

§ 371 Date: Oct. 30, 2023

(30) Foreign Application Priority Data
Apr. 30, 2021 (WO) PCT/US2021/030312
Feb. 8, 2022 (WO) PCT/US2022/015684

(51) Int. Cl. A61K 31/496

(52) U.S. Cl. CPC A61K 31/496

(57) ABSTRACT

Provided herein are safe and effective dosing regimens for mitapivat and/or pharmaceutically acceptable salts thereof.

### RELATED APPLICATIONS

This application claims the benefit of priority to PCT/US2021/030312, filed Apr. 30, 2021 and PCT/US2022/015684, filed Feb. 8, 2022, the entire contents of each of which are incorporated herein by reference.

### BACKGROUND

Pyruvate kinase deficiency (PKD) is a disease of the red blood cells caused by a deficiency of the pyruvate kinase R (PKR) enzyme due to recessive mutations in the PKLR gene. PKR activators can be beneficial to treat PKD, thalassemia (e.g., beta-thalassemia), abetalipoproteinemia or Bassen-Kornzweig syndrome, sickle cell disease, paroxysmal nocturnal hemoglobinuria, anemia (e.g., congenital anemias (e.g., enzymopathies), hemolytic anemia (e.g. hereditary and/or congenital hemolytic anemia, acquired hemolytic anemia, chronic hemolytic anemia caused by phosphoglycerate kinase deficiency, anemia of chronic diseases, non-spherocytic hemolytic anemia or hereditary spherocytosis). Treatment of PKD is supportive, including blood transfusions, splenectomy, chelation therapy to address iron overload, and/or interventions for other disease-related morbidity. Currently, however, there is no approved medicine that treats the underlying cause of PKD, and thus the etiology of life-long hemolytic anemia.

Mitapivat, also known as AG-348 or by its chemical name N-(4-(4-(cyclopropylmethyl)piperazine-1-carbonyl)phenyl)quinoline-8-sulfonamide, is represented by the following structural formula:

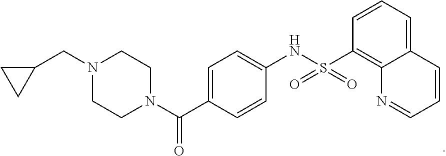 [SMILES: O=C(c1ccc(NS(=O)(=O)c2cccc3cccnc23)cc1)N1CCN(CC2CC2)CC1]

and is a first-in-class, orally bioavailable, potent, allosteric activator of wild-type RBC-specific form of pyruvate kinase (PKR) and a range of PKR-mutant enzymes. See e.g., WO 2011/002817, WO 2016/201227, and Kung et al., Blood. 2017; 130(11):1347-1356. The RBC-specific form of pyruvate kinase is 1 of 4 pyruvate kinase isoenzymes expressed in human tissues from 2 separate genes, liver-specific form of pyruvate kinase (PKL) and pyruvate kinase muscle isozyme (PKM). Both PKR and PKL are splice isoforms of the PKLR gene, while PKM1 and PKM2 are both expressed from the PKM gene. Mitapivat is an allosteric activator of the PKR, PKL, and PKM2 isoenzymes, with similar potency against each.

The hemisulfate sesquihydrate salt of mitapivat, commonly referred to as mitapivat sulfate, has shown positive therapeutic results for the treatment of PKD, thalassemia, and sickle cell disease (SCD). Given its therapeutic potential, means for providing safe and optimized dosing of mitapivat are currently needed.

### SUMMARY

Clinical studies have shown that mitapivat is effective in increasing hemoglobin levels. The extent to which mitapivat elicits its hemoglobin altering affects, however, may vary from subject to subject. That is, the amount of mitapivat required to bring a subject's hemoglobin levels within a desired target range may be lower for one subject and higher for another, or vice versa. Lower than normal levels of hemoglobin can lead to anemia, whereas higher than normal levels can lead to stroke, heart attack, and clots. It is therefore important to provide an optimal dose of mitapivat such that an optimal hemoglobin level is achieved, i.e., not too high and not too low.

Accordingly, provided herein are safe and effective methods for increasing hemoglobin levels in subjects who can benefit from treatment with mitapivat, particularly those with a condition associated with pyruvate kinase, including e.g., PKD, thalassemia, and SCD. In one aspect, the disclosed methods include a) administering an initial amount of mitapivat, or a pharmaceutically acceptable salt thereof, to a subject in need of treatment; b) assessing whether the subject's hemoglobin levels are within a target level; and c) depending on if the subject's hemoglobin levels are at or within the target level, adjusting the amount of mitapivat, or the pharmaceutically acceptable salt thereof, until e.g., the subject's hemoglobin levels are within a target level. This process of assessing and adjusting the amount of mitapivat, or the pharmaceutically acceptable salt thereof, (i.e., steps b and c) may occur multiple times over the course of treatment. In some instances, however a subject's dose of mitapivat or a pharmaceutically acceptable salt thereof, may be adjusted down if the initial amount of mitapivat, or the pharmaceutically acceptable salt thereof, or an adjusted amount of mitapivat or the pharmaceutically acceptable salt thereof, results in an excess level of hemoglobin or too rapid of an increase in a subject's hemoglobin level, or the subject experiences an adverse event.

In one aspect, the methods described herein include a method of treating a condition selected from PKD, thalassemia, or sickle cell disease in a subject in need thereof, the method comprising a) administering to the subject an initial amount of mitapivat or a pharmaceutically acceptable salt thereof for a period of time, wherein the initial amount increases the subject's hemoglobin levels; b) assessing the subject's hemoglobin levels after the period of time to determine whether the subject is within a target hemoglobin level; and c) continuing to administer the initial amount of mitapivat or a pharmaceutically acceptable salt thereof if the subject's hemoglobin level is within the target hemoglobin level, or administering a first adjusted amount of mitapivat, or a pharmaceutically acceptable salt thereof, if the subject's hemoglobin level is not within the target hemoglobin level.

In some aspects, the disclosed methods further comprise continuing to assess the subject's hemoglobin levels and then re-adjusting the amount of mitapivat or a pharmaceutically acceptable salt thereof until the subject's hemoglobin level is within the target hemoglobin level, i.e., steps b and c.

In some aspects, the condition to be treated is selected from thalassemia and sickle cell disease.

In some aspects, the time period between administering an initial and/or adjusted amount of mitapivat, or a pharmaceutically acceptable salt thereof, to the subject and assessing the subject's hemoglobin level ranges from about 1 to about 15 weeks.

In some aspects, the amount of mitapivat administered to the subject as an initial or adjusted amount ranges from about 1 mg to about 125 mg once daily (QD), twice daily (BID), or three times daily (TID). In some aspects, the pharmaceutically acceptable salt of mitapivat administered to the subject as an initial or adjusted amount ranges from an amount equal to about 1 mg mitapivat to about 125 mg mitapivat once daily (QD), twice daily (BID), or three times daily (TID).

In some aspects, the target hemoglobin level is characterized as a normal level of hemoglobin for a male or female subject greater than or equal to about 16 years of age or combination thereof. In some aspects, the target hemoglobin level is characterized as an increase in the subject's baseline hemoglobin level of greater than or equal to about 1.0 g/dL to about 2.5 g/dL over a defined period of time.

Clinical studies have also shown that acute hemolysis with subsequent anemia can occur following abrupt interruption or discontinuation of mitapivat. This result, however, can also vary from subject to subject leaving no reliable means to predict whether or not a subject will have such a reaction. For example, in a phase 2 study with 300 mg of mitapivat dosed twice daily, 3.8% of subjects experienced hemolysis upon sudden withdrawal of mitapivat, including 1 serious adverse event of acute hemolysis. By contrast, patients who missed only a few doses of mitapivat later in their treatment course did not experience events indicative of acute hemolysis. Although the exact risk factors which make someone susceptible to acute hemolysis cannot be determined, it appears that the rapid loss of hemoglobin is an issue for these cases. Given the uncertainty as to who is likely to experience signs or symptoms of acute hemolysis (or subsequent anemia), it is important to develop means for mitigating such events.

Accordingly, also provided herein are methods for discontinuing administration of mitapivat, or a pharmaceutically acceptable salt thereof, or for reducing an established daily amount of mitapivat, or a pharmaceutically acceptable salt thereof, wherein the methods comprise tapering the amount of mitapivat, or a pharmaceutically acceptable salt thereof, and monitoring the subject for indicators of acute hemolysis or anemia, or both, until mitapivat, or a pharmaceutically acceptable salt thereof, is no longer being administered. In some aspects, only one decrease in the dosage amount from the established daily amount of mitapivat, or a pharmaceutically acceptable salt thereof, may be required prior ceasing treatment. In other aspects, multiple reductions in the administered amounts of mitapivat, or a pharmaceutically acceptable salt thereof, are contemplated e.g., in instances where signs and symptoms of acute hemolysis and/or anemia are present, or if the subject is found to be overly susceptible to acute hemolysis and/or anemia. Immediate discontinuance of treatment and subsequent monitoring for acute hemolysis or anemia, or both, may be required in certain instances e.g., in the case of the subject experiencing a clinically significant adverse reaction.

In one aspect, the methods described herein include a method of discontinuing treatment with mitapivat, or a pharmaceutically acceptable salt thereof, as well as a method for reducing the dose of mitapivat, or a pharmaceutically acceptable salt thereof, for a subject being administered an established daily amount of mitapivat or a pharmaceutically acceptable salt thereof, the method comprising a) administering to the subject an adjusted amount of mitapivat or pharmaceutically acceptable salt thereof for a period of time, wherein the first adjusted amount is less than the established daily amount; and b) monitoring the subject for acute hemolysis or anemia, or both, during the period of time.

In another aspect, the methods described herein include a method for reducing the dose of mitapivat, or a pharmaceutically acceptable salt thereof, in a subject being administered an established daily amount of mitapivat or a pharmaceutically acceptable salt thereof, the method comprising a) administering to the subject an adjusted amount of mitapivat or pharmaceutically acceptable salt thereof for a period of time, wherein the adjusted amount is less than the established daily amount; and b) monitoring the subject for acute hemolysis or anemia, or both, during the period of time.

In some aspects, the disclosed methods further comprise continuing to monitor the subject for acute hemolysis or anemia, or both, as well as the subject's hemoglobin levels and then re-adjusting the amount of mitapivat, or a pharmaceutically acceptable salt thereof, until the administration of mitapivat, or a pharmaceutically acceptable salt thereof has ceased. In some aspects, the disclosed methods further comprise continuing to monitor the subject for acute hemolysis or anemia, or both, after the administration of mitapivat, or a pharmaceutically acceptable salt thereof has ceased.

In some aspects, the time periods between administering an initial and/or adjusted amount of mitapivat, or a pharmaceutically acceptable salt thereof, to the subject and monitoring the subject for acute hemolysis and/or anemia ranges from about 1 day to about 20 days.

In some aspects, the amount of mitapivat or pharmaceutically acceptable salt thereof, being administered to the subject as an adjusted amount ranges from about 30% to about 90% of the established daily amount or, in the case of multiple adjusted amounts, from about 30% to about 90% the immediately preceding adjusted amount.

In some aspects, the amount of mitapivat or pharmaceutically acceptable salt thereof, being administered to the subject as an established daily amount or as an adjusted amount ranges from about 1 mg to about 125 mg once daily (QD), twice daily (BID), three times daily (TID), or every other day (QOD).

In some aspects, the subject discontinuing treatment with mitapivat, or a pharmaceutically acceptable salt thereof, was being treated for a condition selected from PKD, thalassemia and sickle cell disease.

In some aspects, the administration of mitapivat, or a pharmaceutically acceptable salt thereof, is immediately ceased (e.g., in the event of the subject suffering a clinically significant adverse reaction) and the subject is monitored for acute hemolysis or anemia, or both, during a period of time.

### DETAILED DESCRIPTION

### Dose Escalation

As part of a first embodiment, provided herein is a method of treating a condition selected from PKD, thalassemia, and sickle cell disease in a subject in need thereof, the method comprising a) administering to the subject an initial amount of mitapivat or a pharmaceutically acceptable salt thereof for a first period of time, wherein the amount increases the subject's hemoglobin levels; b) assessing the subject's hemoglobin levels after the first period of time to determine whether the subject is within a target hemoglobin level; and c) continuing to administer the initial amount of mitapivat or a pharmaceutically acceptable salt thereof if the subject's hemoglobin level is within the target hemoglobin level, or administering a first adjusted amount of mitapivat or a pharmaceutically acceptable salt thereof if the subject's hemoglobin level is not within the target hemoglobin level. Alternatively, as part of a first embodiment, provided herein is a method of treating a condition selected from thalassemia and sickle cell disease in a subject in need thereof, the method comprising a) administering to the subject an initial amount of mitapivat or a pharmaceutically acceptable salt thereof for a first period of time, wherein the amount increases the subject's hemoglobin levels; b) assessing the subject's hemoglobin levels after the first period of time to determine whether the subject is within a target hemoglobin level; and c) continuing to administer the initial amount of mitapivat or a pharmaceutically acceptable salt thereof if the subject's hemoglobin level is within the target hemoglobin level, or administering a first adjusted amount of mitapivat or a pharmaceutically acceptable salt thereof if the subject's hemoglobin level is not within the target hemoglobin level.

As part of a second embodiment, the disclosed methods (such as those in the first embodiment), further comprise administering the first adjusted amount of mitapivat or a pharmaceutically acceptable salt thereof for a second period of time; assessing the subject's hemoglobin levels after the second period of time to determine whether the subject is within the target hemoglobin level; and continuing to administer the first adjusted amount if the subject's hemoglobin level is within the target hemoglobin level, or administering a second adjusted amount of mitapivat or a pharmaceutically acceptable salt thereof, if the subject's hemoglobin level is not within the target hemoglobin level after the second period time.

As part of a third embodiment, the disclosed methods (such as those in the first and second embodiment) further comprise administering the third adjusted amount of mitapivat or a pharmaceutically acceptable salt thereof for a third period of time; or continuing to administer the second adjusted amount if the subject's hemoglobin level is within the target hemoglobin level.

As part of a fourth embodiment, the disclosed methods (such as any one of those in the first to third embodiments) further comprise continuing to assess the subject's hemoglobin levels and re-adjusting the amount of mitapivat or a pharmaceutically acceptable salt thereof until the subject's hemoglobin level is within the target hemoglobin level or continuing to administer mitapivat or a pharmaceutically acceptable salt thereof to the subject without further adjustment.

As part of a fifth embodiment, the periods of times in the disclosed methods (such as any one of those in the first to fourth embodiments) between administering an initial and/or adjusted amount of mitapivat or a pharmaceutically acceptable salt thereof to the subject and assessing the subject's hemoglobin level ranges from about 1 to about 12 weeks and at most about 1 to about 12 weeks. For example, as part of a fifth embodiment, the first, second, and third period of times described herein (such as any one of those in the first to fourth embodiments) are each independently selected from about 1 to about 12 weeks, 1 to about 12 weeks, about 1 to about 11 weeks, about 1 to about 10 weeks, about 1 to about 9 weeks, about 1 to about 8 weeks, about 1 to about 7 weeks, about 1 to about 6 weeks, about 1 to about 5 weeks, about 1 to about 4 weeks, about 1 to about 3 weeks, about 1 to about 2 weeks, about 2 to about 12 weeks, about 2 to about 11 weeks, about 2 to about 10 weeks, about 2 to about 9 weeks, about 2 to about 8 weeks, about 2 to about 7 weeks, about 2 to about 6 weeks, about 2 to about 5 weeks, about 2 to about 4 weeks, about 2 to about 3 weeks, about 3 to about 12 weeks, about 3 to about 11 weeks, about 3 to about 10 weeks, about 3 to about 9 weeks, about 3 to about 8 weeks, about 3 to about 7 weeks, about 3 to about 6 weeks, about 3 to about 5 weeks, about 3 to about 4 weeks, about 4 to about 12 weeks, about 4 to about 11 weeks, about 4 to about 10 weeks, about 4 to about 9 weeks, about 4 to about 8 weeks, about 4 to about 7 weeks, about 4 to about 6 weeks, about 4 to about 5 weeks, about 5 to about 12 weeks, about 5 to about 11 weeks, about 5 to about 10 weeks, about 5 to about 9 weeks, about 5 to about 8 weeks, about 5 to about 7 weeks, about 5 to about 6 weeks, about 6 to about 12 weeks, about 6 to about 11 weeks, about 6 to about 10 weeks, about 6 to about 9 weeks, about 6 to about 8 weeks, and about 6 to about 7 weeks. Alternatively, as part of a fifth embodiment, the first, second, and third period of times described herein (such as any one of those in the first to fourth embodiments) are each independently selected from at least about 1 week, at least about 2 weeks, at least about 3 three weeks, at least about 4 weeks, at least about 5 weeks, at least about 6 weeks, at least about 7 weeks, at least about 8 weeks, at least about 9 weeks, at least about 10 weeks, at least about 11 weeks, and at least about 12 weeks. In another alternative, as part of a fifth embodiment, the first, second, and third period of times described herein (such as any one of those in the first to fourth embodiments) are each independently selected from at most about 1 week, at most about 2 weeks, at most about 3 weeks, at most about 4 weeks, at most about 5 weeks, at most about 6 weeks, at most about 7 weeks, at most about 8 weeks, at most about 9 weeks, at most about 10 weeks, at most about 11 weeks, and at most about 12 weeks. In another alternative, as part of a fifth embodiment, the first, second, and third period of times described herein (such as any one of those in the first to fourth embodiments) are each independently selected from about 1 week, about 2 weeks, about 3 weeks, about 4 weeks, about 5 weeks, about 6 weeks, about 7 weeks, about 8 weeks, about 9 weeks, about 10 weeks, about 11 weeks and about 12 weeks.

As part of a sixth embodiment, certain or all of the periods of times in the disclosed methods (such as any one of those in the first to fifth embodiments) between administering an initial and/or adjusted amount of mitapivat or a pharmaceutically acceptable salt thereof to the subject and assessing the subject's hemoglobin level are of the same duration. For example, as part of a sixth embodiment the first and second period of time described herein (such as any one of those in the first to fifth embodiments) are the same duration; the first and third period of time described herein (such as any one of those in the first to fifth embodiments) are the same duration; the second and third period of time described herein (such as any one of those in the first to fifth embodiments) are the same duration; or the first, second, and third period of time described herein (such as any one of those in the first to fifth embodiments) are all the same duration.

The amount of mitapivat, or a pharmaceutically acceptable salt thereof, used in the disclosed methods is further described herein and can vary depending upon the nature of the method (e.g., dosage or dose escalation vs dosage or dose taper). Here, as part of a seventh embodiment, and e.g., with reference to any one of the first to sixth embodiments, the initial amount of mitapivat, the first adjusted amount of mitapivat, the second adjustment amount of mitapivat, and the third adjusted amount of mitapivat are each independently selected from about 5 mg, about 20 mg, about 50 mg and about 100 mg; and the initial amount of the pharmaceutically acceptable salt of amount of mitapivat, the first adjusted amount of the pharmaceutically acceptable amount of mitapivat, the second adjustment amount of the pharmaceutically acceptable amount of mitapivat, and the third adjusted amount of the pharmaceutically acceptable amount of mitapivat are each independently selected from an amount equal to about 5 mg of mitapivat, about 20 mg of mitapivat, about 50 mg of mitapivat, and about 100 mg of mitapivat. As part of an eighth embodiment, the initial amount, the first adjusted amount, the second adjusted amount, and the third adjusted amount of mitapivat or the pharmaceutically acceptable salt thereof are each administered once daily (QD), twice daily (BID), or three times daily (TID).

As used herein, the term “target hemoglobin level” refers to the normal hemoglobin level of a human based on their age and gender or an rate increase in hemoglobin values that would be sufficient to bring the subject hemoglobin levels to a normal range for a period of time. In one aspect, as part of a ninth embodiment, the target hemoglobin levels in the disclosed methods (e.g., as in any one of the first to eighth embodiments) is characterized as an increase in the subject's baseline hemoglobin level of about 1.0 g/dL to about 2.0 g/dL over any of the first period of time, the second period of time, or the third period of time, individually or collectively. Alternatively, as part of a ninth embodiment, the target hemoglobin levels in the disclosed methods (e.g., as in any one of the first to eighth embodiments) is characterized as an increase in the subject's baseline hemoglobin level of about 1.0 g/dL, about 1.5 g/dL, or about 2.0 g/dL over any of the first period of time, the second period of time, or the third period of time, individually or collectively. In another alternative, as part of a ninth embodiment, the target hemoglobin level in the disclosed methods (e.g., as in any one of the first to eighth embodiments) is between about 12.0 g/dL to about 17.5 g/dL. In another alternative, as part of a ninth embodiment, the target hemoglobin level in the disclosed methods (e.g., as in any one of the first to eighth embodiments) is between about 12.0 g/dL to about 17.5 g/dL for subject's ≥16 years of age. In another alternative, as part of a ninth embodiment, the target hemoglobin level in the disclosed methods (e.g., as in any one of the first to eighth embodiments) is between about 13.5 g/dL to about 17.5 g/dL for adult males (≥18 years of age) and between about 12.0 g/dL to about 15.5 g/dL for adult (≥18 years of age) females. In another alternative, as part of a ninth embodiment, the target hemoglobin level in the disclosed methods (e.g., as in any one of the first to eighth embodiments) is between about 13.5 g/dL to about 17.5 g/dL for males ≥16 years of age and between about 12.0 g/dL to about 15.5 g/dL for females ≥16 years of age). In another alternative, as part of a ninth embodiment, the target hemoglobin level in the disclosed methods (e.g., as in any one of the first to eighth embodiments) is between about 11.9 g/dL to about 15.0 g/dL for female subjects age 11 to 18. In another alternative, as part of a ninth embodiment, the target hemoglobin level in the disclosed methods (e.g., as in any one of the first to eighth embodiments) is between about 12.7 g/dL to about 17.7 g/dL for male subjects age 11 to 18. In another alternative, as part of a ninth embodiment, the target hemoglobin level in the disclosed methods (e.g., as in any one of the first to eighth embodiments) is between about 10.7 g/dL to about 19.9 g/dL for subjects less than 18 years old (i.e., non-adult subjects).

As used herein, a “subject's baseline hemoglobin level” refers to the subject's hemoglobin level prior to administering mitapivat or a pharmaceutically acceptable salt thereof e.g., prior to the administration of the initial amount of mitapivat or a pharmaceutically acceptable salt thereof in the foregoing embodiments. In some aspects, a subject's baseline hemoglobin level is less than about 14.0 g/dL such as e.g., less than about 13.5 g/dL for an adult male and less than about 12.0 g/dL for an adult female. In other aspects, a subject's baseline hemoglobin level is between about 5.5 g/dL to about 10.5 g/dL.

### Dose Tapering

As part of a tenth embodiment, provided herein is a method of discontinuing treatment with mitapivat or a pharmaceutically acceptable salt thereof for a subject being administered an established daily amount of mitapivat or a pharmaceutically acceptable salt thereof, the method comprising a) administering to the subject a first adjusted amount of mitapivat or pharmaceutically acceptable salt thereof for a first period of time, wherein the first adjusted amount is less than the established daily amount; and b) monitoring the subject for acute hemolysis or anemia, or both, during the first period of time. Alternatively, as part of a tenth embodiment, provided herein is a method for reducing an established daily amount of mitapivat or a pharmaceutically acceptable salt thereof being administered to a subject, the method comprising a) administering to the subject a first adjusted amount of mitapivat or pharmaceutically acceptable salt thereof for a first period of time, wherein the first adjusted amount is less than the established daily amount; and b) monitoring the subject for acute hemolysis or anemia, or both, during the first period of time.

As used herein, “the established daily amount” of mitapivat or a pharmaceutically acceptable salt thereof and “the established daily dosage” of mitapivat or a pharmaceutically acceptable salt thereof are synonymous and refer to the amount of mitapivat or pharmaceutically acceptable that is being administered to the subject prior to administering to the subject the first adjusted amount of mitapivat or pharmaceutically acceptable salt thereof. As further described herein, the established daily amount can range from about 1 mg to about 300 mg of mitapivat or a pharmaceutically acceptable salt of mitapivat in an amount equal to about 1 mg to about 300 mg of mitapivat. In some embodiments, the established daily amount can range from about 1 mg to about 200 mg of mitapivat or a pharmaceutically acceptable salt of mitapivat in an amount equal to about 1 mg to about 200 mg of mitapivat.

As part of an eleventh embodiment, the disclosed methods (e.g., as in the tenth embodiment) further comprise the step of: c) treating the subject for acute hemolysis or anemia, or both, if the subject shows symptoms of acute hemolysis or anemia, or both.

As part of a twelfth embodiment, the disclosed methods (e.g., as in the eleventh embodiment) comprises repeating step a) and step b) using a second adjusted amount for a second period of time and continuing to monitor the subject for symptoms of acute hemolysis or anemia or both, wherein the second adjusted amount is less than the first adjusted amount.

As part of a thirteenth embodiment, the disclosed methods (e.g., as in any one of the tenth to eleventh and thirty-fourth and thirty-sixth embodiments) further comprises the step of treating the subject for acute hemolysis or anemia, or both, if the subject shows symptoms of acute hemolysis or anemia, or both.

As part of a fourteenth embodiment, administration of the first adjusted amount in the disclosed methods (e.g., as in any one of the tenth to thirteenth embodiments) is continued until the symptoms of acute hemolysis or anemia, or both, improve.

As part of a fifteenth embodiment, administration of the second adjusted amount in the disclosed methods (e.g., as in any one of the twelfth to fourteenth embodiments) is initiated immediately after completion of the first period of time.

As part of a sixteenth embodiment, the disclosed methods (e.g., as in any one of the twelfth to fifteenth embodiments) further comprise repeating step a) and step b) using a third adjusted amount for a third period of time and continuing to monitor the subject for symptoms of acute hemolysis or anemia, or both, wherein the third adjusted amount is less than the adjusted amount administered in the previous step a). Alternatively, as part of a sixteenth embodiment, the disclosed methods (e.g., as in any one of the twelfth to fifteenth embodiments) further comprise repeating step a) and step b) using a third adjusted amount for a third period of time and continuing to monitor the subject for symptoms of acute hemolysis or anemia, or both, wherein the third adjusted amount is less than the adjusted amount administered in the previous step a), and wherein administration of the third adjusted amount is initiated immediately after completion of the second period of time.

As part of a seventeenth embodiment, step a) and b) in the disclosed methods (e.g., as in any one of the tenth to sixteenth embodiments) are repeated until the subject is no longer being administered mitapivat or a pharmaceutically acceptable salt thereof.

As part of an eighteenth embodiment, the disclosed methods (e.g., as in any one of the tenth to seventeenth and thirty-fourth and thirty-sixth embodiments) further comprise the step of treating the subject for acute hemolysis and/or anemia, if the subject shows symptoms of acute hemolysis and/or anemia. Alternatively, as part of an eighteenth embodiment, the disclosed methods (e.g., as in any one of the tenth to seventeenth and thirty-fourth and thirty-sixth embodiments) further comprise the step of treating the subject for acute hemolysis and/or anemia, if the subject shows symptoms of acute hemolysis and/or anemia; and continuing to treat the subject until the symptoms of acute hemolysis or anemia, or both, improve.

As part of a nineteenth embodiment, the established daily amount of mitapivat being administered in the disclosed methods (e.g., as in any one of the tenth to eighteenth embodiments) to the subject prior to the first adjusted amount is selected from about 10 mg per day, about 40 mg per day, about 100 mg per day, and about 200 mg per day; and the established daily amount of the pharmaceutically acceptable salt is selected from an amount equal to about 10 mg of mitapivat per day, about 40 mg of mitapivat per day, about 100 mg of mitapivat per day, and about 200 mg per day of mitapivat per day. Alternatively, as part of a nineteenth embodiment, the established daily amount of mitapivat being administered in the disclosed methods (e.g., as in any one of the tenth to eighteenth embodiments) to the subject prior to the first adjusted amount is selected from about 5 mg BID, about 20 mg BID, about 50 mg BID or about 100 mg BID; and the established daily amount of the pharmaceutically acceptable salt being administered to the subject prior to the first adjusted amount is equal to about 5 mg BID of mitapivat, about 20 mg BID of mitapivat, about 50 mg BID of mitapivat, or about 100 mg BID of mitapivat.

As part of a twentieth embodiment, the first adjusted amount of mitapivat or the pharmaceutically acceptable salt in the disclosed methods (e.g., as in any one of the tenth to nineteenth embodiments) is an amount selected from about 40% to about 80%, from about 40% to about 60%, from about 45% to about 55%, and from about 49% to about 51% less than the established daily amount of mitapivat or the pharmaceutically acceptable salt. Alternatively, as part of a twentieth embodiment, the first adjusted amount of mitapivat or the pharmaceutically acceptable salt in the disclosed methods (e.g., as in any one of the tenth to nineteenth embodiments) is about 50% less than the established daily amount of mitapivat or the pharmaceutically acceptable salt. In another alternative, as part of a twentieth embodiment, the first adjusted amount of mitapivat or the pharmaceutically acceptable salt in the disclosed methods (e.g., as in any one of the tenth to nineteenth embodiments) is selected from about 5 mg, about 20 mg, about 50 mg, and about 100 mg; and the first adjusted amount of the pharmaceutically acceptable salt is equal to about 5 mg of mitapivat, about 20 mg mitapivat, about 50 mg mitapivat, and about 100 mg mitapivat. In another alternative, as part of a twentieth embodiment, the first adjusted amount of mitapivat or the pharmaceutically acceptable salt in the disclosed methods (e.g., as in any one of the tenth to nineteenth embodiments) is selected from about 5 mg QD, about 20 mg QD, about 50 mg QD, and about 100 mg QD; and the first adjusted amount of the pharmaceutically acceptable salt is equal to about 5 mg QD of mitapivat, about 20 mg QD mitapivat, about 50 mg QD mitapivat, and about 100 mg QD mitapivat.

As part of a twenty-first embodiment, the second adjusted amount of mitapivat or the pharmaceutically acceptable salt in the disclosed methods (e.g., as in any one of the tenth to twentieth embodiments) reduces the first adjusted amount of mitapivat or the pharmaceutically acceptable salt in an amount selected from about 60% to about 80%, from about 70% to about 80%, from about 45% to about 85%, from about 55% to about 65% less than the established daily amount of mitapivat or the pharmaceutically acceptable salt. Alternatively, as part of a twenty-first embodiment, the second adjusted amount of mitapivat or the pharmaceutically acceptable salt in the disclosed methods (e.g., as in any one of the tenth to twentieth embodiments) is about 60% or about 75% less than the established daily amount of mitapivat or the pharmaceutically acceptable salt thereof. In another alternative, as part of a twenty-first embodiment, the second adjusted amount of mitapivat or the pharmaceutically acceptable salt in the disclosed methods (e.g., as in any one of the tenth to twentieth embodiments) is about 50 mg, about 20 mg, or about 5 mg; and the second adjusted amount of the pharmaceutically acceptable salt of mitapivat is equal to about 50 mg of mitapivat, about 20 mg of mitapivat, or about 5 mg of mitapivat.

As part of a twenty-second embodiment, the second adjusted amount of mitapivat or a pharmaceutically acceptable salt thereof in the disclosed methods (e.g., as in any one of the tenth to twenty-first embodiments) is equal to the amount of the first adjusted amount of mitapivat or the pharmaceutically acceptable salt administered every other day (QOD). Alternatively, as part of a twenty-second embodiment, the second adjusted amount of mitapivat or a pharmaceutically acceptable salt thereof in the disclosed methods (e.g., as in any one of the tenth to twenty-first embodiments) is about 5 mg QOD, about 20 mg QOD, about 20 mg QD or about 50 mg QD; and the second adjusted amount of the pharmaceutically acceptable salt is equal to about 5 mg QOD of mitapivat, about 20 mg QOD of mitapivat, about 20 mg QD of mitapivat, or about 50 mg QD of mitapivat.

As part of a twenty-third embodiment, the third adjusted amount of mitapivat or the pharmaceutically acceptable salt in the disclosed methods (e.g., as in any one of the sixteenth to twenty-first embodiments) is equal to the amount of the second adjusted amount of mitapivat or the pharmaceutically acceptable salt administered every other day (QOD). Alternatively, as part of a twenty-third embodiment, the third adjusted amount of mitapivat in the disclosed methods (e.g., as in any one of the sixteenth to twenty-first embodiments) is about 20 mg QOD or about 50 mg QOD; and the third adjusted amount of the pharmaceutically acceptable salt is equal to about 20 mg QOD of mitapivat or about 50 mg QOD of mitapivat.

As part of a twenty-fourth embodiment, the periods of time between administering and monitoring (such as any of those in the tenth to twenty-third embodiments) are each independently selected from about 1 day to about 16 days. Alternatively, for example, as part of a twenty-fourth embodiment, the first and second period of time in the disclosed methods (such as any of those in the tenth to twenty-third embodiments) are each independently selected from about 1 day to about 14 days, from about 1 day to about 12 days, from about 1 day to about 10 days, from about 2 days to about 14 days, from about 2 days to about 12 days, from about 2 days to about 10 days, from about 2 days to about 9 days, from about 2 days to about 5 days, from about 2 days to about 4 days, from about 5 days to about 9 days, from about 5 days to about 8 days, and from about 6 days to about 8 days. In another alternative, as part of a twenty-fourth embodiment, the first and second period of time in the disclosed methods (such as any of those in the tenth to twenty-third embodiments) are each independently selected from about 2 days to about 5 days, from about 2 days to about 4 days, from about 5 days to about 8 days, and from about 6 days to about 8 days. In another alternative, as part of a twenty-fourth embodiment, the first and second period of time in the disclosed methods (such as any of those in the tenth to twenty-third embodiments) are each independently selected from about 3 days and about 7 days.

As part of a twenty-fifth embodiment, the first and second period of time in the disclosed methods (such as any of those in the tenth to twenty-fourth embodiments) are both the same.

As part of a twenty-sixth embodiment, the third period of time in the disclosed methods (such as any of those in the sixteenth to twenty-fifth embodiments) is selected from about 1 day to about 14 days, from about 1 day to about 12 days, from about 1 day to about 10 days, from about 2 days to about 14 days, from about 2 days to about 12 days, from about 2 days to about 10 days, from about 2 days to about 9 days, from about 2 days to about 5 days, from about 2 days to about 4 days, from about 5 days to about 9 days, from about 5 days to about 8 days, and from about 6 days to about 8 days. Alternatively, as part of a twenty-sixth embodiment, the third period of time in the disclosed methods (such as any of those in the sixteenth to twenty-fifth embodiments) is selected from about 2 days to about 5 days, from about 2 days to about 4 days, from about 5 days to about 8 days, and from about 6 days to about 8 days. In another alternative, as part of a twenty-sixth embodiment, the third period of time in the disclosed methods (such as any of those in the sixteenth to twenty-fifth embodiments) is about 7 days.

As part of a twenty-seventh embodiment, the first, second, and third period of time in the disclosed methods (such as any of those in the tenth to twenty-sixth embodiments) are each the same duration.

As part of twenty-eighth embodiment, the disclosed methods (such as any of those in the tenth to twenty-seventh and thirty-fourth and thirty-sixth embodiments) further comprise monitoring the subject for acute hemolysis or anemia, or both, after ceasing the administration of mitapivat or a pharmaceutically acceptable salt thereof. Alternatively, as part of twenty-eight embodiment, the disclosed methods (such as any of those in the tenth to twenty-seventh and thirty-fourth and thirty-sixth embodiments) further comprise monitoring the subject for acute hemolysis or anemia, or both, after ceasing the administration of mitapivat or a pharmaceutically acceptable salt thereof, wherein monitoring comprises testing the subject for acute hemolysis or anemia, or both.

As part of a twenty-ninth embodiment, provided is a method for discontinuing administration of an established daily amount of mitapivat or a pharmaceutically acceptable salt thereof to a subject in need thereof, the method comprising a) immediately ceasing all administration of mitapivat or a pharmaceutically acceptable salt thereof; and b) monitoring the subject for acute hemolysis or anemia, or both, during a period of time (e.g., about 1 day to about 14 days, from about 1 day to about 12 days, from about 1 day to about 10 days, from about 2 days to about 14 days, from about 2 days to about 12 days, from about 2 days to about 10 days, from about 2 days to about 9 days, from about 2 days to about 5 days, from about 2 days to about 4 days, from about 5 days to about 9 days, from about 5 days to about 8 days, from about 6 days to about 8 days, for about 14 days or less, or for about 7 days or less).

As part of a thirtieth embodiment, provided is a method for discontinuing administration of an established daily dose of mitapivat or a pharmaceutically acceptable salt thereof to a subject suffering from an adverse event, the method comprising a) immediately ceasing all administration of mitapivat or a pharmaceutically acceptable salt thereof; and b) monitoring the subject for acute hemolysis or anemia, or both, during a period of time (e.g., about 1 day to about 14 days, from about 1 day to about 12 days, from about 1 day to about 10 days, from about 2 days to about 14 days, from about 2 days to about 12 days, from about 2 days to about 10 days, from about 2 days to about 9 days, from about 2 days to about 5 days, from about 2 days to about 4 days, from about 5 days to about 9 days, from about 5 days to about 8 days, from about 6 days to about 8 days, for about 14 days or less, or for about 7 days or less). Alternatively, as part of a thirtieth embodiment, provided is a method for discontinuing administration of an established daily dose of mitapivat or a pharmaceutically acceptable salt thereof to a subject suffering from a clinically significant adverse reaction, the method comprising a) immediately ceasing all administration of mitapivat or a pharmaceutically acceptable salt thereof; and b) monitoring the subject for acute hemolysis or anemia, or both, during a period of time (e.g., about 1 day to about 14 days, from about 1 day to about 12 days, from about 1 day to about 10 days, from about 2 days to about 14 days, from about 2 days to about 12 days, from about 2 days to about 10 days, from about 2 days to about 9 days, from about 2 days to about 5 days, from about 2 days to about 4 days, from about 5 days to about 9 days, from about 5 days to about 8 days, from about 6 days to about 8 days, for about 14 days or less, or for about 7 days or less).

Further provided herein is a method for administering mitapivat or a pharmaceutically acceptable salt thereof to a subject, whereby the abrupt discontinuation of mitapivat or a pharmaceutically acceptable salt thereof is avoided to minimize the risk of acute hemolysis.

As used herein, “acute hemolysis” refers to a rupturing of red blood cells which can lead to hemolytic anemia. Methods for determining if a subject is experiencing acute hemolysis are known in the art and include both physical and visual symptoms. In one aspect, for example, as part of a thirty-first embodiment, acute hemolysis as disclosed in the present methods (such as any of those in the tenth to thirtieth and thirty-sixth embodiments) is characterized by a rapid loss in hemoglobin of greater than about 0.1 g/dL/day to about 0.2 g/dL/day. Alternatively, as part of a thirty-first embodiment, acute hemolysis as disclosed in the present methods (such as any of those in the tenth to thirtieth and thirty-sixth embodiments) is characterized by a loss in hemoglobin of from about 1.7 g/dL/day to about 2.5 g/dL/day. In another alternative, as part of a thirty-first embodiment, acute hemolysis as disclosed in the present methods (such as any of those in the tenth to thirtieth and thirty-sixth embodiments) is characterized by the subject having one or more of jaundice, scleral icterus and dark urine. In another alternative, as part of a thirty-first embodiment, acute hemolysis as disclosed in the present methods (such as any of those in the tenth to thirtieth and thirty-sixth embodiments) is characterized by the subject having one or more of darkening of the urine, yellowing of the skin or eyes, dizziness, confusion, feeling tired, light-headedness, or shortness of breath. In another alternative, as part of a thirty-first embodiment, acute hemolysis as disclosed in the present methods (such as any of those in the tenth to thirtieth and thirty-sixth embodiments) is characterized by the subject having one or more of jaundice, scleral icterus, dark urine, dizziness, confusion, fatigue, or shortness of breath.

As part of a thirty-second embodiment, the subject in the disclosed methods (e.g., as in any one of the tenth to thirty-first and thirty-fourth and thirty-sixth embodiments) is being treated for a condition selected from pyruvate kinase deficiency (PKD), thalassemia, and sickle cell disease (SCD). Alternatively, as part of a thirty-second embodiment, the subject in the disclosed methods (e.g., as in any one of the tenth to thirty-first and thirty-fourth and thirty-sixth embodiments) is being treated for PKD. In another alternative, as part of a thirty-second embodiment, the subject in the disclosed methods (e.g., as in any one of the tenth to thirty-first and thirty-fourth and thirty-sixth embodiments) is being treated for hemolytic anemia or anemia caused by or associated with pyruvate kinase deficiency (PKD). In another alternative, as part of a thirty-second embodiment, the subject in the disclosed methods (e.g., as in any one of the tenth to thirty-first and thirty-fourth and thirty-sixth embodiments) is being treated for SCD. In still another alternative, the subject in the disclosed methods (e.g., as in any one of the tenth to thirty-first and thirty-fourth and thirty-sixth embodiments) is being treated for thalassemia. In a further alternative, the subject in the disclosed methods (e.g., as in any one of the tenth to thirty-first and thirty-fourth and thirty-sixth embodiments) is being treated for either alpha thalassemia or beta thalassemia.

As part of a thirty-third embodiment, the administration of mitapivat or a pharmaceutically acceptable salt thereof as disclosed herein (e.g., as in any one of the tenth to thirty-second embodiments) is ceased immediately, regardless of the time period or established daily dose, if the subject is suffering from an adverse event or a clinically significant adverse reaction.

In one aspect, the thalassemia of the disclosed methods (e.g., as in any one of the first to thirty-third embodiments) is non-transfusion dependent thalassemia or transfusion dependent thalassemia.

In one aspect, the thalassemia of the disclosed methods (e.g., as in any one of the first to thirty-third embodiments) is alpha thalassemia or beta thalassemia.

As part of a thirty-fourth embodiment, provided herein is a method of discontinuing treatment with mitapivat or a pharmaceutically acceptable salt thereof (e.g., a sulfate salt, hemisulfate salt or hemisulfate sesquihydrate salt) for a subject being administered about 5 mg of mitapivat twice daily (BID) or an amount of a pharmaceutically acceptable salt (e.g., a sulfate salt, hemisulfate salt or hemisulfate sesquihydrate salt) of mitapivat that is equal to about 5 mg of mitapivat BID, the method comprising administering mitapivat or a pharmaceutically acceptable thereof (e.g., a sulfate salt, hemisulfate salt or hemisulfate sesquihydrate salt) according to the following dose taper schedule: Day 1 to Day 7: administering about 5 mg of mitapivat once daily (QD) to the subject or administering an amount of a pharmaceutically acceptable salt of mitapivat (e.g., a sulfate salt, hemisulfate salt or hemisulfate sesquihydrate salt) that is equal to about 5 mg of mitapivat QD to the subject; Day 8: discontinuing the administration of mitapivat or the pharmaceutically acceptable salt of mitapivat (e.g., a sulfate salt, hemisulfate salt or hemisulfate sesquihydrate salt) thereafter. Alternatively, as part of a thirty-fourth embodiment, provided herein is a method of discontinuing treatment with mitapivat or a pharmaceutically acceptable salt thereof (e.g., a sulfate salt, hemisulfate salt or hemisulfate sesquihydrate salt) for a subject being administered about 5 mg of mitapivat twice daily (BID) or an amount of a pharmaceutically acceptable salt of mitapivat (e.g., a sulfate salt, hemisulfate salt or hemisulfate sesquihydrate salt) that is equal to about 5 mg of mitapivat BID, the method comprising administering mitapivat or a pharmaceutically acceptable thereof (e.g., a sulfate salt, hemisulfate salt or hemisulfate sesquihydrate salt) according to the following dose taper schedule: Day 1 to Day 7: administering about 5 mg of mitapivat once daily (QD) to the subject or administering an amount of a pharmaceutically acceptable salt of mitapivat (e.g., a sulfate salt, hemisulfate salt or hemisulfate sesquihydrate salt) that is equal to about 5 mg of mitapivat QD to the subject; Day 8 to Day 14: administering about 5 mg of mitapivat every other day (QOD) to the subject or administering an amount of a pharmaceutically acceptable salt of mitapivat (e.g., a sulfate salt, hemisulfate salt or hemisulfate sesquihydrate salt) that is equal to about 5 mg of mitapivat QOD to the subject; Day 15: discontinuing the administration of mitapivat or the pharmaceutically acceptable salt thereof (e.g., a sulfate salt, hemisulfate salt or hemisulfate sesquihydrate salt). In another alternative, as part of a thirty-fourth embodiment, provided herein is a method of discontinuing treatment with mitapivat or a pharmaceutically acceptable salt thereof (e.g., a sulfate salt, hemisulfate salt or hemisulfate sesquihydrate salt) for a subject being administered about 20 mg of mitapivat twice daily (BID) or an amount of a pharmaceutically acceptable salt of mitapivat (e.g., a sulfate salt, hemisulfate salt or hemisulfate sesquihydrate salt) that is equal to about 20 mg of mitapivat BID, the method comprising administering mitapivat or a pharmaceutically acceptable thereof (e.g., a sulfate salt, hemisulfate salt or hemisulfate sesquihydrate salt) according to the following dose taper schedule: Day 1 to Day 7: administering about 20 mg of mitapivat once daily (QD) to the subject or administering an amount of a pharmaceutically acceptable salt of mitapivat (e.g., a sulfate salt, hemisulfate salt or hemisulfate sesquihydrate salt) that is equal to about 20 mg of mitapivat QD to the subject; Day 8 to Day 14: administering about 5 mg of mitapivat once daily (QD) to the subject or administering an amount of a pharmaceutically acceptable salt of mitapivat (e.g., a sulfate salt, hemisulfate salt or hemisulfate sesquihydrate salt) that is equal to about 5 mg of mitapivat QD to the subject; Day 15: discontinuing the administration of mitapivat or the pharmaceutically acceptable salt thereof (e.g., a sulfate salt, hemisulfate salt or hemisulfate sesquihydrate salt). In another alternative, as part of a thirty-fourth embodiment, provided herein is a method of discontinuing treatment with mitapivat or a pharmaceutically acceptable salt thereof (e.g., a sulfate salt, hemisulfate salt or hemisulfate sesquihydrate salt) for a subject being administered about 20 mg of mitapivat twice daily (BID) or an amount of a pharmaceutically acceptable salt of mitapivat (e.g., a sulfate salt, hemisulfate salt or hemisulfate sesquihydrate salt) that is equal to about 20 mg of mitapivat BID to the subject, the method comprising administering mitapivat or a pharmaceutically acceptable thereof (e.g., a sulfate salt, hemisulfate salt or hemisulfate sesquihydrate salt) according to the following dose taper schedule: Day 1 to Day 7: administering about 20 mg of mitapivat once daily (QD) to the subject or administering an amount of a pharmaceutically acceptable salt of mitapivat (e.g., a sulfate salt, hemisulfate salt or hemisulfate sesquihydrate salt) that is equal to about 20 mg of mitapivat QD to the subject; Day 8 to Day 14: administering about 20 mg of mitapivat every other day (QOD) to the subject or administering an amount of a pharmaceutically acceptable salt of mitapivat (e.g., a sulfate salt, hemisulfate salt or hemisulfate sesquihydrate salt) that is equal to about 20 mg of mitapivat QOD to the subject; Day 15: discontinuing the administration of mitapivat or the pharmaceutically acceptable salt thereof (e.g., a sulfate salt, hemisulfate salt or hemisulfate sesquihydrate salt). In another alternative, as part of a thirty-fourth embodiment, provided herein is a method of discontinuing treatment with mitapivat or a pharmaceutically acceptable salt thereof (e.g., a sulfate salt, hemisulfate salt or hemisulfate sesquihydrate salt) for a subject being administered about 50 mg of mitapivat twice daily (BID) or administering an amount of a pharmaceutically acceptable salt of mitapivat (e.g., a sulfate salt, hemisulfate salt or hemisulfate sesquihydrate salt) that is equal to about 50 mg of mitapivat BID to the subject, the method comprising administering mitapivat or a pharmaceutically acceptable thereof (e.g., a sulfate salt, hemisulfate salt or hemisulfate sesquihydrate salt) according to the following dose taper schedule: Day 1 to Day 7: administering about 50 mg of mitapivat once daily (QD) to the subject or administering an amount of a pharmaceutically acceptable salt of mitapivat (e.g., a sulfate salt, hemisulfate salt or hemisulfate sesquihydrate salt) that is equal to about 50 mg of mitapivat QD to the subject; Day 8 to Day 14: administering about 20 mg of mitapivat once daily (QD) to the subject or administering an amount of a pharmaceutically acceptable salt of mitapivat (e.g., a sulfate salt, hemisulfate salt or hemisulfate sesquihydrate salt) that is equal to about 20 mg of mitapivat QD to the subject; Day 15: discontinuing the administration of mitapivat or the pharmaceutically acceptable salt thereof (e.g., a sulfate salt, hemisulfate salt or hemisulfate sesquihydrate salt). In another alternative, as part of a thirty-fourth embodiment, provided herein is a method of discontinuing treatment with mitapivat or a pharmaceutically acceptable salt thereof (e.g., a sulfate salt, hemisulfate salt or hemisulfate sesquihydrate salt) for a subject being administered about 50 mg of mitapivat twice daily (BID) or administering an amount of a pharmaceutically acceptable salt of mitapivat (e.g., a sulfate salt, hemisulfate salt or hemisulfate sesquihydrate salt) that is equal to about 50 mg of mitapivat BID to the subject, the method comprising administering mitapivat or a pharmaceutically acceptable thereof (e.g., a sulfate salt, hemisulfate salt or hemisulfate sesquihydrate salt) according to the following dose taper schedule: Day 1 to Day 7: administering about 50 mg of mitapivat once daily (QD) to the subject or administering an amount of a pharmaceutically acceptable salt of mitapivat (e.g., a sulfate salt, hemisulfate salt or hemisulfate sesquihydrate salt) that is equal to about 50 mg of mitapivat QD to the subject; Day 8 to Day 14: administering about 20 mg of mitapivat once daily (QD) to the subject or administering an amount of a pharmaceutically acceptable salt of mitapivat (e.g., a sulfate salt, hemisulfate salt or hemisulfate sesquihydrate salt) that is equal to about 20 mg of mitapivat QD to the subject; Day 15 to Day 21 administering about 20 mg of mitapivat every other day (QOD) to the subject or administering an amount of a pharmaceutically acceptable salt of mitapivat (e.g., a sulfate salt, hemisulfate salt or hemisulfate sesquihydrate salt) that is equal to about 20 mg of mitapivat QOD to the subject; Day 22: discontinuing the administration of mitapivat or the pharmaceutically acceptable salt thereof (e.g., a sulfate salt, hemisulfate salt or hemisulfate sesquihydrate salt).

In a thirty-fifth embodiment, the methods described in the thirty-fourth embodiment, further comprise monitoring the subject for acute hemolysis or anemia, or both.

In a thirty-sixth embodiment, the mitapivat or pharmaceutically acceptable salt thereof described in the above dosage taper may be crystalline or amorphous.

Throughout this application, parentheticals are used to further describe certain aspects of the invention and are not to be construed as limitations. For example, with reference to salts, the terms “sulfate salt”, “hemisulfate salt”, and “hemisulfate sesquihydrate salt” as referred to in “or the pharmaceutically acceptable salt of mitapivat (e.g., a sulfate salt, hemisulfate salt or hemisulfate sesquihydrate salt)” are provided as alternative exemplary embodiments and are not to be read as limiting descriptions of the broader term “salts”.

The terms “treatment,” “treat,” and “treating” refer to reversing, alleviating, reducing the likelihood of developing, or inhibiting the progress of a disease or disorder, or one or more effects or symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of genetic or other susceptibility factors), i.e., prophylactic treatment. Treatment may also be continued after symptoms have resolved, for example to reduce the likelihood of or delay their recurrence.

As used herein the terms “subject” and “patient” may be used interchangeably, and means a mammal in need of or undergoing treatment. Typically, the subject is a human in need of treatment. In some aspects, the subject in the disclosed methods is an adult human subject (i.e., a male or female human of 18 years of age or greater). In other aspects, the subject in the disclosed methods is non-adult human subject (i.e., a male or female human of 17 years of age or less). In other aspects, the subject in the disclosed methods is a male or female human 16 years of age or greater.

The terms “dose” and “dosage” are used interchangeably.

As used herein the term “adverse reaction” means an undesirable effect, reasonably associated with the use of drug that may occur as part of the pharmacological action of the drug or may be unpredictable in its occurrence.

As used herein the term “clinically significant adverse reaction” means any reaction in a patient or subject occurring at any dose that results in any of the following outcomes: death, a life-threatening adverse event, impatient hospitalization or prolongation of existing hospitalization, a persistent or significant disability or incapacity, or a congenital anomaly or birth defect. Other medical events that may not result in death or be life threatening, or require hospitalization may be considered an adverse reaction when, based on appropriate medical judgement, such events may jeopardize the patient or subject and may require medical or surgical intervention to prevent any one of the outcomes listed in the preceding sentence.

As used herein the term “adverse event” means any untoward medical event associated with the use of a drug in humans, whether or not the medical event is considered to be drug related.

In some aspects, mitapivat or a pharmaceutically acceptable salt thereof, as disclosed in the present methods (e.g., as in any one of the first to thirty-fourth embodiments) may be crystalline or amorphous.

The term “pharmaceutically acceptable salt” when referring to a pharmaceutically acceptable salt of mitapivat, refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art, for example, Berge et al. describe pharmaceutically acceptable salts in detail in J. Pharmaceutical Sciences, 1977, 66, 1-19. Pharmaceutically acceptable salts of mitapivat include those derived from suitable inorganic and organic acids. Examples of pharmaceutically acceptable acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, besylate bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, gentisate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, tosylate p-toluenesulfonate, undecanoate, valerate salts, and the like.

In some aspects, the pharmaceutically acceptable salt of mitapivat as described in the present methods (e.g., as in any one of the first to thirty-fourth embodiments) is a sulfate salt. In some aspects, the pharmaceutically acceptable salt of mitapivat as described in the present methods (e.g., as in any one of the first to thirty-fourth embodiments) is a hemisulfate salt. In some aspects, the pharmaceutically acceptable salt of mitapivat as described in the present methods (e.g., as in any one of the first to thirty-fourth embodiments) is a hydrated hemisulfate salt. In some aspects, the pharmaceutically acceptable salt of mitapivat as described in the present methods (e.g., as in any one of the first to thirty-fourth embodiments) is a hemisulfate sesquihydrate salt, also known as mitapivat sulfate or 1-(cyclopropylmethyl)-4-(4-(quinoline-8-sulfonamido)benzoyl)piperazin-1-ium hemisulfate sesquihydrate having Formula A:

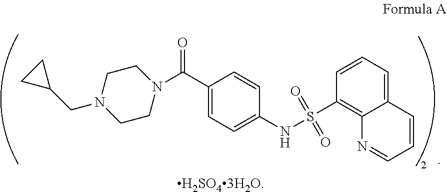

Alternatively, in some aspects, the pharmaceutically acceptable salt of mitapivat as described in the present methods (e.g., as in any one of the first to thirty-fourth embodiments) is a sulfate trihydrate salt, also referred to as mitapivat trihydrate or 1-(cyclopropylmethyl)-4-(4-(quinoline-8-sulfonamido)benzoyl)piperazin-1-ium sulfate trihydrate having Formula B:

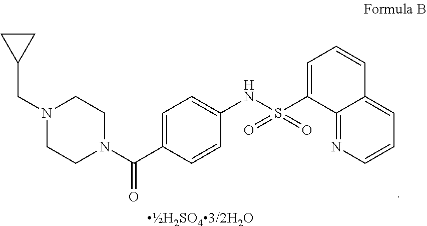

The hemisulfate sesquihydrate salt of mitapivat (i.e., mitapivat sulfate) can be crystalline, for example, Form A as disclosed in U.S. Publication No. 20200277279. Form A is characterized by one or more of the following x-ray powder diffraction patterns at 20 angles (±0.2°) using Cu Kα radiation: 9.9°, 15.8°, and 22.6°; 15.0°, 17.1°, 21.3°, and 21.9°; 9.9°, 15.0°, 15.8°, 17.1°, 21.3°, 21.9°, and 22.6°; 9.9°, 11.4°, 15.0°, 15.3°, 15.8°, 17.1°, 17.7°, 21.3°, 21.9°, 22.6°, and 23.5°; or 4.9°, 9.9°, 11.0°, 11.4°, 11.7°, 12.3°, 12.8°, 13.6°, 13.9°, 14.2°, 15.0°, 15.3°, 15.8°, 17.1°, 17.4°, 17.7°, 18.8°, 19.1°, 19.8°, 21.3°, 21.9°, 22.6°, 23.0°, 23.2°, 23.5°, 23.8°, 24.1°, 24.5°, 25.3°, 25.6°, 26.1°, 27.1°, 28.1°, and 29.8°. In some embodiments, Form A is characterized by x-ray powder diffraction peaks at 20 angles (±0.2°) 9.9°, 15.8°, and 22.6°. In certain embodiments, Form A is characterized by x-ray powder diffraction peaks at 20 angles (±0.2°) 9.9°, 15.8°, and 22.6° and at least one additional x-ray powder diffraction peak at 20 angles (±0.2°) selected from 15.0°, 17.10, 21.3°, and 21.9°. In certain embodiments, Form A is characterized by x-ray powder diffraction peaks at 20 angles (±0.2°) 9.9°, 15.8°, and 22.6°; and at least two additional x-ray powder diffraction peaks at 20 angles (±0.2°) selected from 15.0°, 17.1°, 21.3°, and 21.9°. In yet another alternative, Form A is characterized by x-ray powder diffraction peaks at 20 angles (±0.2°) 9.9°, 15.8°, and 22.6°; and at least three additional x-ray powder diffraction peaks at 20 angles (±0.2°) selected from 15.0°, 17.1°, 21.3°, and 21.9°. In certain embodiments, Form A is characterized by x-ray powder diffraction peaks at 20 angles (±0.2°) 9.9°, 15.0°, 15.8°, 17.1°, 21.3°, 21.9°, and 22.6°. In certain embodiments, Form A is characterized by x-ray powder diffraction peaks at 20 angles (±0.2°) 9.9°, 11.4°, 15.0°, 15.3°, 15.8°, 17.1°, 17.7°, 21.3°, 21.9°, 22.6°, and 23.5°. In certain embodiments, Form A is characterized by x-ray powder diffraction peaks at 20 angles (±0.2°) 4.9°, 9.9°, 11.0°, 11.4°, 11.7°, 12.3°, 12.8°, 13.6°, 13.9°, 14.2°, 15.0°, 15.3°, 15.8°, 17.1°, 17.4°, 17.7°, 18.8°, 19.1°, 19.8°, 21.3°, 21.9°, 22.6°, 23.0°, 23.2°, 23.5°, 23.8°, 24.1°, 24.5°, 25.3°, 25.6°, 26.1°, 27.1°, 28.1°, and 29.8°. In yet another alternative, Form A is characterized by a differential scanning calorimetry (DSC) thermograph comprising endotherm peaks at about 159° C.±5° C. and 199° C.±5° C. In yet another alternative, crystalline Form A is characterized by a thermogravimetric analysis (TGA) thermogram comprising a weight loss of about 4.5±0.5% up to 180° C.±2° C. In some embodiments, the hemisulfate sesquihydrate salt of mitapivat is 1-(cyclopropylmethyl)-4-(4-(quinoline-8-sulfonamido)benzoyl)piperazin-1-ium hemisulfate sesquihydrate Form A.

As used herein, the dose amount of mitapivat or a pharmaceutically acceptable salt thereof (e.g., the hemisulfate sesquihydrate of mitapivat) is based on the equivalence to the free-base form of mitapivat. For example, the hemisulfate sesquihydrate salt of mitapivat in the disclosed methods in an amount equal to about 2.0 mg of mitapivat means about 2.0 mg of free-base mitapivat or about 2.33 mg of the hemisulfate sesquihydrate salt having the structural formula A.

Mitapivat, or a pharmaceutically acceptable salt thereof (e.g., mitapivat sulfate), may be formulated and administered as a pharmaceutical composition. Pharmaceutical compositions of mitapivat or a pharmaceutically acceptable salt thereof (e.g., mitapivat sulfate) can be prepared by any method known in the art of pharmacology. In general, such preparatory methods include the steps of bringing of mitapivat or a pharmaceutically acceptable salt thereof (e.g., mitapivat sulfate) into association with a carrier and/or one or more other accessory ingredients, and then, if necessary and/or desirable, shaping and/or packaging the product into a desired single- or multi-dose unit. In one aspect, the pharmaceutical compositions are orally administered in an orally acceptable dosage form including, but not limited to, capsules, tablets, emulsions and aqueous suspensions, dispersions and solutions. In one aspect, mitapivat or a pharmaceutically acceptable salt thereof (e.g. mitapivat sulfate) is formulated as a tablet composition together with a pharmaceutically acceptable carrier, in accordance with the disclosures of International Patent Application No. WO 2019/104134.

In some aspects, mitapivat or a pharmaceutically acceptable salt thereof (e.g. mitapivat sulfate) is formulated in the form of taper packs. In some aspects, mitapivat or a pharmaceutically acceptable salt thereof (e.g. mitapivat sulfate) is formulated as about 5 mg, about 20 mg, about 50 mg or about 100 mg taper packs. In some aspects, mitapivat or a pharmaceutically acceptable salt thereof (e.g. mitapivat sulfate) is formulated as about 5 mg, about 20 mg, about 50 mg or about 100 mg taper packs packaged in cartons. In other aspects, mitapivat or a pharmaceutically acceptable salt thereof (e.g. mitapivat sulfate) is formulated as about 5 mg, about 20 mg, about 50 mg or about 100 mg taper packs packaged in cartons comprising at least 1 blister card (or wallet). In other aspects, mitapivat or a pharmaceutically acceptable salt thereof (e.g. mitapivat sulfate) is formulated as taper packs packaged in cartons comprising at least 1 blister card (or wallet) containing from about 5 (five) to about 10 (ten) dosage forms of about 5 mg, about 20 mg, about 50 mg or about 100 mg of mitapivat. In other aspects, the about 5 mg dosage forms are round, blue, film coated tablets with “M5” printed on one side. In other aspects, the about 20 mg dosage forms are round, blue, film coated tablets with “M20” printed on one side. In other aspects, the about 50 mg dosage forms are oblong, blue, film coated tablets with “M50” printed on one side. In other aspects, the about 100 mg dosage forms are film coated tablets with “M100” printed or embossed on one side. In some aspects, mitapivat or a pharmaceutically acceptable salt thereof (e.g. mitapivat sulfate) is formulated as follows:
about 5 mg taper packs packaged in cartons containing 1 blister card (or wallet) with 7 round, blue, film-coated tablets with “M5” printed on one side.
about 5 mg taper packs packaged in cartons containing 2 blister cards (or wallets) with 7 round, blue, film-coated tablets with “M5” printed on one side.
about 20 mg taper packs packaged in cartons containing 2 blister cards (or wallets) with 7 round, blue, film-coated tablets with “M20” printed on one side.
about 50 mg taper packs packaged in cartons containing 2 blister cards (or wallets) with 7 oblong, blue, film-coated tablets with “M50” printed on one side.
about 100 mg taper packs packaged in cartons containing 2 blister cards (or wallets) with 7 film-coated tablets with “M100” printed or embossed on one side.

As used herein, the terms “about” and “approximately” are used herein to mean within the typical ranges of tolerance in the art. In one embodiment, “about” means within 2 standard deviations from the mean value. In one embodiment, “about” means±10%. In one embodiment, “about” means±5%. In another embodiment, “about” means±2 days. When “about” is present before a series of numbers of number ranges, it is understood that the term applies to any and each of the numbers and ranges recited in the series.

### EXEMPLIFICATION

### Example 1: Dosage Escalation and Tapering for Subjects with Pyruvate Kinase Deficiency (PKD)

To gradually increase hemoglobin (Hb) levels and maximize the therapeutic effect, mitapivat should be titrated through sequential doses of 5 mg, 20 mg, and 50 mg twice daily with dose increases to the next dose level every 4 weeks (see Table 1). Hb level should be assessed before increasing to the next dose level as some patients may reach normal Hb levels at 5 mg or 20 mg twice daily. The maximum recommended dose is 50 mg twice daily.

TABLE 1
Dose Titration Schedule

| Duration | Dose Titration |
|---|---|
| Day 1 to Week 4 | 5 mg twice daily |

| Assess Hb level | | If Hb level is within |
|---|---|---|
| every 4 weeks | If Hb level is below normal range | normal range |
| Week 5 to Week 8 | Increase to 20 mg twice daily and | Maintain 5 mg twice |
| | maintain for 4 weeks. | daily. |
| Week 9 to Week 12 | Prior dose 5 mg twice daily: | Maintain current dose |
| | Increase to 20 mg dose twice daily | (5 mg twice daily or 20 |
| | and maintain for 4 weeks. | mg twice daily). |
| | Prior dose 20 mg twice daily: | |
| | Increase to 50 mg dose twice daily | |
| | and maintain for 4 weeks. | |
| Week 13 to | Prior dose 5 mg twice daily: | Maintain current dose |
| Maintenance | Consider increasing to 20 mg dose | (5 mg twice daily, 20 |
| | twice daily. | mg twice daily or 50 |
| | Prior dose 20 mg twice daily: | mg twice daily). |
| | Consider increasing to 50 mg dose | |
| | twice daily. | |
| | Prior dose 50 mg twice daily: | |
| | Maintain dose at 50 mg twice daily. | |

If a dose reduction is required then reduce to the next lowest dose level, 20 mg twice daily or 5 mg twice daily.

To minimize the risk of acute hemolysis, abrupt interruption or discontinuation of mitapivat should be avoided. Instead, the dosage of mitapivat should be tapered e.g., following the schedule in Table 2 or Table 2A. Patients should also be monitored for signs of acute hemolysis with anemia including jaundice, scleral icterus, dark urine, dizziness, confusion, fatigue, and/or shortness of breath during the tapering period.

TABLE 2
Dose Taper Schedule

| | Dose Taper Schedule | | |
|---|---|---|---|
| Current Dose | Day 1-7 | Day 8-14 | Day 15 |
| 5 mg twice daily | 5 mg once daily | Discontinue | N/A |
| 20 mg twice daily | 20 mg once daily | 5 mg once daily | Discontinue |
| 50 mg twice daily | 50 mg once daily | 20 mg once daily | Discontinue |

Abbreviations:
N/A = not applicable.

TABLE 2A
Alternative Dose Taper Schedule

| | Dose Taper Schedule | | | |
|---|---|---|---|---|
| Current Dose | Day 1-7 | Day 8-14 | Day 15-21 | Day 22 |
| 5 mg twice | 5 mg once | 5 mg every | Discontinue | N/A |
| daily | daily | other day | | |
| 20 mg twice | 20 mg once | 20 mg every | Discontinue | N/A |
| daily | daily | other day | | |
| 50 mg twice | 50 mg once | 20 mg once | 20 mg every | Discontinue |
| daily | daily | daily | other day | |

Abbreviations:
N/A = not applicable.

### Example 2—Phase 3, Double-Blind, Randomized, Placebo-Controlled, Multicenter Study Evaluating the Efficacy and Safety of Mitapivat in Adult Subjects with Non-Transfusion-Dependent Alpha- or Beta-Thalassemia (ENERGIZE

The primary objective of the study is to compare the effect of mitapivat versus placebo on anemia (i.e., Hb response), supported by patient-reported outcome (FACIT-Fatigue) and performance outcome (6MWT) measures evaluating how subjects feel and function, and hemolytic and erythropoietic parameters and iron metabolism. Other secondary objectives include the evaluation of pharmacokinetic and pharmacodynamic parameters and safety. Safety will be evaluated by the incidence, severity, and type of AEs, and by evaluation of vital signs, physical examination findings, clinical laboratory results, and bone mineral density scans. Transfusions and other supportive care therapies (including iron chelation therapy) are permitted as clinically indicated to manage symptoms and prevent secondary complications. Dose modifications are permitted under certain conditions, for example in the event of excessive Hb response.

Inclusion Criteria for subjects include e.g.:
≥18 years of age at the time of providing informed consent.
Documented diagnosis of thalassemia (β-thalassemia with or without α-globin gene mutations, HbE/β-thalassemia, or α-thalassemia/HbH disease) based on Hb electrophoresis, Hb high-performance liquid chromatography, and/or DNA analysis from the subject's medical record. If this information is not available from the subject's medical record, the test(s) can be performed by a local laboratory during the Screening Period. If a local laboratory is unable to perform the test(s), results from the comprehensive α- and β-globin genotyping performed by the study central laboratory can be used.
Hb concentration ≤10.0 g/dL, based on an average of at least 2 Hb concentration measurements (separated by ≥7 days) collected during the Screening Period.
Non-transfusion dependent, defined as ≤5 red blood cell (RBC) units during the 24-week period before randomization, and no RBC transfusions ≤8 weeks before providing informed consent or during the Screening Period.
If taking hydroxyurea, the hydroxyurea dose must be stable for ≥16 weeks before randomization.

The primary endpoint of the study is hemoglobin (Hb) response, defined as a ≥1.0 g/dL increase in average Hb concentration from Week 12 through Week 24 compared with baseline.

Subjects will receive 100 mg BID mitapivat or matched placebo for oral administration. Subjects who discontinue the study will undergo dose tapering and will be monitored for signs and symptoms of acute hemolysis and worsening of anemia. Exemplarily dosage tapering protocols, based on various starting dosage of mitapivat, are described below.

TABLE 3
Study 1 Dose Taper

| Dose | Schedule |
|---|---|
| 100 mg | BID |
| 100 mg | QD for 7 days, then discontinue mitapivat |

TABLE 4
Rapid Dose Taper

| Starting | First Step × | Second Step × | | Third Step × |
|---|---|---|---|---|
| Dose | 3 days | 3 days | | 3 days |
| 5 mg BID | 5 mg QD | 5 | mg QOD | — |
| 20 mg BID | 20 mg QD | 20 | mg QOD | — |
| 50 mg BID | 50 mg QD | 20 | mg QD | 20 mg QOD |

TABLE 5
Gradual Dose Taper

| Starting | First Step × | Second Step × | | Third Step × |
|---|---|---|---|---|
| Dose | 7 days | 7 days | | 7 days |
| 5 mg BID | 5 mg QD | 5 | mg QOD | — |
| 20 mg BID | 20 mg QD | 20 | mg QOD | — |
| 50 mg BID | 50 mg QD | 20 | mg QD | 20 mg QOD |

TABLE 6
Study 2 Dose Taper

| Starting | First Step × | Second Step × |
|---|---|---|
| Dose | 7 days | 7 days |
| 5 mg BID | 5 mg QD | — |
| 20 mg BID | 20 mg QD | 5 mg QD |
| 50 mg BID | 50 mg QD | 20 mg QD |

TABLE 6A
Alternative Study 2 Dose Taper

| Starting | First Step × | Second Step × | | Third Step × |
|---|---|---|---|---|
| Dose | 7 days | 7 days | | 7 days |
| 5 mg BID | 5 mg QD | 5 | mg QOD | — |
| 20 mg BID | 20 mg QD | 20 | mg QOD | — |
| 50 mg BID | 50 mg QD | 20 | mg QD | 20 mg QOD |

Dosage escalation, e.g., as described in Example 1, is optional for this study.

### Example 3—Phase 3, Double-Blind, Randomized, Placebo-Controlled, Multicenter Study Evaluating the Efficacy and Safety of Mitapivat in Adult Subjects with Transfusion-Dependent Alpha- or Beta-Thalassemia (ENERGIZE-T

The primary objective of the study is to compare the effect of mitapivat versus placebo on transfusion burden in subjects with α- or β-transfusion dependent thalassemia (TDT). Other secondary objectives include the evaluation of markers of iron overload, pharmacokinetic and pharmacodynamic parameters, and safety. Safety will be evaluated by the incidence, severity, and type of AEs, and by evaluation of vital signs, physical examination findings, clinical laboratory results, and bone mineral density scans. Subjects will continue to receive appropriate supportive care to manage symptoms and prevent secondary complications as clinically indicated and according to applicable guidelines. Dose modifications are permitted for excessive hemoglobin response, study drug-related AEs, and adverse events of special interest of transaminase increases.

Inclusion Criteria for subjects include e.g.:
≥18 years of age at the time of providing informed consent.
Documented diagnosis of thalassemia (β-thalassemia with or without α-globin gene mutations, HbE/β-thalassemia, or α-thalassemia/HbH disease) based on Hb electrophoresis, Hb high-performance liquid chromatography, and/or DNA analysis from the subject's medical record. If this information is not available from the subject's medical record, the test(s) can be performed by a local laboratory during the Screening Period. If a local laboratory is unable to perform the test(s), results from the comprehensive α- and β-globin genotyping performed by the study central laboratory can be used.
Transfusion dependent, defined as 6 to 20 RBC units transfused and a ≤6-week transfusion-free period during the 24-week period before randomization.
If taking hydroxyurea, the hydroxyurea dose must be stable for ≥16 weeks before randomization.

The primary endpoint of the study is transfusion reduction response (TRR), defined as a ≥50% reduction in transfused red blood cell (RBC) units with a reduction of ≥2 units of transfused RBCs in any consecutive 12-week period through Week 48 compared with baseline.

Subjects will receive 100 mg BID mitapivat or matched placebo for oral administration. Subjects who discontinue the study will undergo dose tapering and will be monitored for signs and symptoms of acute hemolysis and worsening of anemia. Exemplary dosage tapering protocols, based on various starting dosage of mitapivat, are described above in Table 3-6. Dosage escalation, e.g., as described in Example 1, is optional for this study.

### Numbered Embodiments

Embodiment 1. A method of treating thalassemia or sickle cell disease in a subject in need thereof, the method comprising administering to the subject an initial amount of mitapivat or a pharmaceutically acceptable salt thereof for a first period of time, wherein the amount increases the subject's hemoglobin levels; assessing the subject's hemoglobin levels after the first period of time to determine whether the subject is within a target hemoglobin level; and continuing to administer the initial amount of mitapivat or a pharmaceutically acceptable salt thereof if the subject's hemoglobin level is within the target hemoglobin level or administering a first adjusted amount of mitapivat or a pharmaceutically acceptable salt thereof if the subject's hemoglobin level is not within the target hemoglobin level.

Embodiment 2. The method of Embodiment 1, wherein the method further comprises administering the first adjusted amount of mitapivat or a pharmaceutically acceptable salt thereof for a second period of time; assessing the subject's hemoglobin levels after the second period of time to determine whether the subject is within the target hemoglobin level; and continuing to administer the first adjusted amount if the subject's hemoglobin level is within the target hemoglobin level or administering a second adjusted amount of mitapivat or a pharmaceutically acceptable salt thereof, if the subject's hemoglobin level is not within the target hemoglobin level after the second period time.

Embodiment 3. The method of Embodiment 1 or 2, wherein the method further comprises administering a third adjusted amount of mitapivat or a pharmaceutically acceptable salt thereof for a third period of time; or continuing to administer the second adjusted amount if the subject's hemoglobin level is within the target hemoglobin level.

Embodiment 4. The method of any one of Embodiments 1 to 3, wherein the method further comprises continuing to assess the subject's hemoglobin levels and re-adjusting the amount of mitapivat or a pharmaceutically acceptable salt thereof until the subject's hemoglobin level is within the target hemoglobin level, or continuing to administer mitapivat or a pharmaceutically acceptable salt thereof to the subject without further adjustment.

Embodiment 5. The method of any one of Embodiments 1 to 4, wherein the first, second, and third period of time are each independently selected from about 1 to about 12 weeks, about 1 to about 11 weeks, about 1 to about 10 weeks, about 1 to about 9 weeks, about 1 to about 8 weeks, about 1 to about 7 weeks, about 1 to about 6 weeks, about 1 to about 5 weeks, about 1 to about 4 weeks, about 1 to about 3 weeks, about 1 to about 2 weeks, about 2 to about 12 weeks, about 2 to about 11 weeks, about 2 to about 10 weeks, about 2 to about 9 weeks, about 2 to about 8 weeks, about 2 to about 7 weeks, about 2 to about 6 weeks, about 2 to about 5 weeks, about 2 to about 4 weeks, about 2 to about 3 weeks, about 3 to about 12 weeks, about 3 to about 11 weeks, about 3 to about 10 weeks, about 3 to about 9 weeks, about 3 to about 8 weeks, about 3 to about 7 weeks, about 3 to about 6 weeks, about 3 to about 5 weeks, about 3 to about 4 weeks, about 4 to about 12 weeks, about 4 to about 11 weeks, about 4 to about 10 weeks, about 4 to about 9 weeks, about 4 to about 8 weeks, about 4 to about 7 weeks, about 4 to about 6 weeks, about 4 to about 5 weeks, about 5 to about 12 weeks, about 5 to about 11 weeks, about 5 to about 10 weeks, about 5 to about 9 weeks, about 5 to about 8 weeks, about 5 to about 7 weeks, about 5 to about 6 weeks, about 6 to about 12 weeks, about 6 to about 11 weeks, about 6 to about 10 weeks, about 6 to about 9 weeks, about 6 to about 8 weeks, and about 6 to about 7 weeks.

Embodiment 6. The method of any one of Embodiments 1 to 4, wherein the first, second, and third period of time are each independently selected from at least about 1 week, at least about 2 weeks, at least about 3 three weeks, at least about 4 weeks, at least about 5 weeks, at least about 6 weeks, at least about 7 weeks, at least about 8 weeks, at least about 9 weeks, at least about 10 weeks, at least about 11 weeks, and at least about 12 weeks.

Embodiment 7. The method of any one of Embodiments 1 to 4, wherein the first, second, and third period of time are each independently selected from at most about 1 week, at most about 2 weeks, at most about 3 weeks, at most about 4 weeks, at most about 5 weeks, at most about 6 weeks, at most about 7 weeks, at most about 8 weeks, at most about 9 weeks, at most about 10 weeks, at most about 11 weeks, and at most about 12 weeks.

Embodiment 8. The method of any one of Embodiments 1 to 4, wherein the first, second, and third period of time are each independently selected from about 1 week, about 2 weeks, about 3 weeks, about 4 weeks, about 5 weeks, about 6 weeks, about 7 weeks, about 8 weeks, about 9 weeks, about 10 weeks, about 11 weeks and about 12 weeks.

Embodiment 9. The method of any one of Embodiments 1 to 8, wherein the first and second period of time are the same duration; the first and third period of time are the same duration; the second and third period of time are the same duration; or the first, second, and third period of time are all the same duration.

Embodiment 10. The method of any one of Embodiments 1 to 9, wherein the initial amount of mitapivat, the first adjusted amount of mitapivat, the second adjustment amount of mitapivat, and the third adjusted amount of mitapivat are each independently selected from about 5 mg, about 20 mg, about 50 mg and about 100 mg; and wherein the initial amount of the pharmaceutically acceptable salt of amount of mitapivat, the first adjusted amount of the pharmaceutically acceptable amount of mitapivat, the second adjustment amount of the pharmaceutically acceptable amount of mitapivat, and the third adjusted amount of the pharmaceutically acceptable amount of mitapivat are each independently selected from an amount equal to about 5 mg of mitapivat, about 20 of mitapivat, about 50 mg of mitapivat, and about 100 mg of mitapivat.

Embodiment 11. The method of any one of Embodiments 1 to 10, wherein the initial amount, the first adjusted amount, the second adjusted amount, and the third adjusted amount of mitapivat or the pharmaceutically acceptable salt thereof are each administered one daily (QD), twice daily (BID), or three times daily (TID).

Embodiment 12. The method of any one of Embodiments 1 to 11, wherein the target hemoglobin level is characterized as an increase in the subject's baseline hemoglobin level of about 1.0 g/dL to about 2.0 g/dL over any of the first period of time, the second period of time, or the third period of time, individually or collectively.

Embodiment 13. The method of any one of Embodiments 1 to 12, wherein the target hemoglobin level is characterized as an increase in the subject's baseline hemoglobin level of about 1.0 g/dL, about 1.5 g/dL, or about 2.0 g/dL over any of the first period of time, the second period of time, or the third period of time, individually or collectively.

Embodiment 14. The method of any one of Embodiments 1 to 11, wherein the target hemoglobin level is between about 12.0 g/dL to about 17.5 g/dL.

Embodiment 15. The method of any one of Embodiments 1 to 11 and 14, wherein the target hemoglobin level is between about 13.5 g/dL to about 17.5 g/dL for males ≥16 years of age and between about 12.0 g/dL to about 15.5 g/dL for females ≥16 years of age.

Embodiment 16. The method of any one of Embodiments 1 to 15, wherein the thalassemia is non-transfusion dependent thalassemia or transfusion dependent thalassemia.

Embodiment 17. The method of any one of Embodiments 1 to 16, wherein the thalassemia is selected from non-transfusion dependent alpha thalassemia, non-transfusion dependent beta thalassemia, transfusion dependent alpha thalassemia, or transfusion dependent beta thalassemia.

Embodiment 18. A method of discontinuing treatment with mitapivat or a pharmaceutically acceptable salt thereof for a subject being administered an established daily amount of mitapivat or a pharmaceutically acceptable salt thereof, the method comprising a) administering to the subject a first adjusted amount of mitapivat or pharmaceutically acceptable salt thereof for a first period of time, wherein the first adjusted amount is less than the established daily amount; and b) monitoring the subject for acute hemolysis or anemia, or both, during the first period of time.

Embodiment 19. A method for reducing an established daily amount of mitapivat or a pharmaceutically acceptable salt thereof being administered to a subject, the method comprising a) administering to the subject a first adjusted amount of mitapivat or pharmaceutically acceptable salt thereof for a first period of time, wherein the first adjusted amount is less than the established daily amount; and b) monitoring the subject for acute hemolysis or anemia, or both, during the first period of time.

Embodiment 20. The method of Embodiment 18 or 19, further comprising the step of: c) treating the subject for acute hemolysis or anemia, or both, if the subject shows symptoms of acute hemolysis or anemia, or both.

Embodiment 21. The method of Embodiment 20, comprising repeating step a) and step b) using a second adjusted amount of mitapivat or a pharmaceutically acceptable salt thereof for a second period of time and continuing to monitor the subject for symptoms of acute hemolysis or anemia, or both, wherein the second adjusted amount is less than the first adjusted amount.

Embodiment 22. The method of Embodiment 21, further comprising the step of treating the subject for acute hemolysis or anemia, or both, if the subject shows symptoms of acute hemolysis or anemia, or both.

Embodiment 23. The method of any one of Embodiments 20 to 22, wherein administration of the first adjusted amount of mitapivat or a pharmaceutically acceptable salt thereof is continued until the symptoms of acute hemolysis or anemia, or both, improve.

Embodiment 24. The method of any one of Embodiments 21 to 23, wherein administration of the second adjusted amount of mitapivat or a pharmaceutically acceptable salt thereof is initiated immediately after completion of the first period of time.

Embodiment 25. The method of any one of Embodiments 21 to 24, further comprising repeating step a) and step b) using a third adjusted amount of mitapivat or a pharmaceutically acceptable salt thereof for a third period of time and continuing to monitor the subject for symptoms of acute hemolysis or anemia or both, wherein the third adjusted amount is less than the adjusted amount administered in the previous step a).

Embodiment 26. The method of Embodiment 25, wherein administration of the third adjusted amount of mitapivat or a pharmaceutically acceptable salt thereof is initiated immediately after completion of the second period of time.

Embodiment 27. The method of any one of Embodiments 18 to 26, wherein step a) and b) are repeated until the subject is no longer being administered mitapivat or a pharmaceutically acceptable salt thereof.

Embodiment 28. The method of any one of Embodiments 18 to 27, further comprising the step of treating the subject for acute hemolysis or anemia, or both, if the subject shows symptoms of acute hemolysis or anemia, or both.

Embodiment 29. The method of Embodiment 28, wherein treating is continued until the symptoms of acute hemolysis or anemia, or both, improve.

Embodiment 30. The method of any one of Embodiments 18 to 29, wherein the established daily amount of mitapivat is selected from about 10 mg per day, about 40 mg per day, about 100 mg per day, and about 200 mg per day; and wherein the established daily amount of the pharmaceutically acceptable salt is selected from an amount equal to about 10 mg of mitapivat per day, about 40 mg of mitapivat per day, about 100 mg of mitapivat per day, and about 200 mg of mitapivat per day.

Embodiment 31. The method of any one of Embodiments 18 to 30, wherein the established daily amount of mitapivat being administered to the subject prior to the first adjusted amount is selected from about 5 mg BID, about 20 mg BID, about 50 mg BID or about 100 mg BID; and wherein the established daily amount of the pharmaceutically acceptable salt being administered to the subject prior to the first adjusted amount is equal to about 5 mg BID of mitapivat, about 20 mg BID of mitapivat, about 50 mg BID of mitapivat, or about 100 mg BID of mitapivat.

Embodiment 32. The method of any one of Embodiments 18 to 31, wherein the first adjusted amount of mitapivat or the pharmaceutically acceptable salt is an amount selected from about 40% to about 80%, from about 40% to about 60%, from about 45% to about 55%, and from about 49% to about 51% less than the established daily amount of mitapivat or the pharmaceutically acceptable salt.

Embodiment 33. The method of any one of Embodiments 18 to 32, wherein the first adjusted amount of mitapivat or the pharmaceutically acceptable salt is about 50% less than the established daily amount of mitapivat or the pharmaceutically acceptable salt.

Embodiment 34. The method of any one of Embodiments 18 to 33, wherein the first adjusted amount of mitapivat is selected from about 5 mg, about 20 mg, about 50 mg, and about 100 mg; and wherein the first adjusted amount of the pharmaceutically acceptable salt is equal to about 5 mg of mitapivat, about 20 mg mitapivat, about 50 mg mitapivat, and about 100 mg mitapivat.

Embodiment 35. The method of any one of Embodiments 18 to 34, wherein the first adjusted amount of mitapivat is selected from about 5 mg QD, about 20 mg QD, about 50 mg QD, and about 100 mg QD; and wherein the first adjusted amount of the pharmaceutically acceptable salt is equal to about 5 mg QD of mitapivat, about 20 mg QD mitapivat, about 50 mg QD mitapivat, and about 100 mg QD mitapivat.

Embodiment 36. The method of any one of Embodiments 21 to 35, wherein the second adjusted amount of mitapivat or the pharmaceutically acceptable salt reduces the first adjusted amount of mitapivat or the pharmaceutically acceptable salt in an amount selected from about 60% to about 80%, from about 70% to about 80%, from about 45% to about 85%, from about 55% to about 65% less than the established daily amount of mitapivat or the pharmaceutically acceptable salt.

Embodiment 37. The method of any one of Embodiments 19 to 36, wherein the second adjusted amount of mitapivat or the pharmaceutically acceptable salt is about 60% or about 75% less than the established daily amount of mitapivat or a pharmaceutically acceptable salt thereof.

Embodiment 38. The method of any one of Embodiments 19 to 37, wherein the second adjusted amount of mitapivat is about 50 mg, about 20 mg, or about 5 mg; and wherein the second adjusted amount of the pharmaceutically acceptable salt of mitapivat is equal to about 50 mg of mitapivat, about 20 mg of mitapivat, or about 5 mg of mitapivat.

Embodiment 39. The method of any one of Embodiments 19 to 38, wherein the second adjusted amount of mitapivat or the pharmaceutically acceptable salt is equal to the amount of the first adjusted amount of mitapivat or the pharmaceutically acceptable salt administered once every day (QD).

Embodiment 40. The method of Embodiment 39, wherein the amount of the second adjusted amount of mitapivat is about 5 mg QOD, about 20 mg QOD, about 20 mg QD or about 50 mg QD; and wherein the second adjusted amount of the pharmaceutically acceptable salt is equal to about 5 mg QOD of mitapivat, about 20 mg QOD of mitapivat, about 20 mg QOD of mitapivat, or about 50 mg QD of mitapivat.

Embodiment 41. The method of any one of Embodiments 25 to 40, wherein the third adjusted amount of mitapivat or the pharmaceutically acceptable salt is equal to the amount of the second adjusted amount of mitapivat or the pharmaceutically acceptable salt administered every other day (QOD).

Embodiment 42. The method of any one of Embodiments 23 to 41, wherein the third adjusted amount of mitapivat is about 20 mg QOD or about 50 mg QOD; and wherein the third adjusted amount of the pharmaceutically acceptable salt is equal to about 20 mg QOD or about 50 mg QOD of mitapivat.

Embodiment 43. The method of any one of Embodiments 16 to 42, wherein the first and second period of time are each independently selected from about 1 day to about 14 days, from about 1 day to about 12 days, from about 1 day to about 10 days, from about 2 days to about 14 days, from about 2 days to about 12 days, from about 2 days to about 10 days, from about 2 days to about 9 days, from about 2 days to about 5 days, from about 2 days to about 4 days, from about 5 days to about 9 days, from about 5 days to about 8 days, and from about 6 days to about 8 days.

Embodiment 44. The method of any one of Embodiments 18 to 43, wherein the first and second period of time are each independently selected from about 2 days to about 5 days, from about 2 days to about 4 days, from about 5 days to about 8 days, and from about 6 days to about 8 days.

Embodiment 45. The method of any one of Embodiments 18 to 44, wherein the first and second period of time are each independently selected from about 3 days and about 7 days.

Embodiment 46. The method of any one of Embodiments 18 to 45, wherein the first and second period of time are both the same duration.

Embodiment 47. The method of any one of Embodiments 25 to 46, wherein the third period of time is selected from about 1 day to about 14 days, from about 1 day to about 12 days, from about 1 day to about 10 days, from about 2 days to about 14 days, from about 2 days to about 12 days, from about 2 days to about 10 days, from about 2 days to about 9 days, from about 2 days to about 5 days, from about 2 days to about 4 days, from about 5 days to about 9 days, from about 5 days to about 8 days, and from about 6 days to about 8 days.

Embodiment 48. The method of any one of Embodiments 25 to 47, wherein the third period of time is selected from about 2 days to about 5 days, from about 2 days to about 4 days, from about 5 days to about 8 days, and from about 6 days to about 8 days.

Embodiment 49. The method of any one of Embodiments 25 to 48, wherein the third period of time is about 7 days.

Embodiment 50. The method of any one of Embodiments 25 to 49, wherein the first, second, and third period of time are each the same duration.

Embodiment 51. The method of any one of Embodiments 18 to 50, further comprising monitoring the subject for acute hemolysis or anemia, or both after ceasing the administration of mitapivat or a pharmaceutically acceptable salt thereof.

Embodiment 52. The method of any one of Embodiments 18 to 51, wherein monitoring comprises testing the subject for acute hemolysis or anemia, or both.

Embodiment 53. The method of any one of Embodiments 18 to 52, wherein acute hemolysis is characterized by a rapid loss in hemoglobin.

Embodiment 54. The method of any one of Embodiments 18 to 53, wherein acute hemolysis is characterized by a loss in hemoglobin of greater than about 0.1 g/dL/day to about 0.2 g/dL/day.

Embodiment 55. The method of any one of Embodiments 18 to 54, wherein acute hemolysis is characterized by a loss in hemoglobin of from about 1.7 g/dL/day to about 2.5 g/dL/day.

Embodiment 56. The method of any one of Embodiments 18 to 52, wherein acute hemolysis or anemia, or both, is characterized as the subject having one or more of jaundice, scleral icterus, dark urine, dizziness, confusion, fatigue, or shortness of breath.

Embodiment 57. The method of any one of Embodiments 18 to 56, wherein the administration of mitapivat or a pharmaceutically acceptable salt thereof is ceased immediately, regardless of the time period or established daily dose, if the subject is suffering from an adverse event.

Embodiment 58. The method of Embodiments 18 to 57, wherein the subject is being treated for a condition selected from pyruvate kinase deficiency (PKD), thalassemia, and sickle cell disease (SCD).

Embodiment 59. The method of Embodiments 18 to 57, wherein the subject is being treated for hemolytic anemia or anemia caused by or associated with pyruvate kinase deficiency (PKD).

Embodiment 60. The method of Embodiments 16 to 58, wherein the subject is being treated for sickle cell disease (SCD).

Embodiment 61. The method of embodiment 58, wherein the thalassemia is non-transfusion dependent thalassemia or transfusion dependent thalassemia.

Embodiment 62. The method of Embodiment 58 or 61, wherein the thalassemia is alpha thalassemia or beta thalassemia.

Embodiment 63. A method for discontinuing administration of an established daily dose of mitapivat or a pharmaceutically acceptable salt thereof to a subject in need thereof, the method comprising a) immediately ceasing all administration of mitapivat or a pharmaceutically acceptable salt thereof; and b) monitoring the subject for acute hemolysis or anemia, or both, during a period of time.

Embodiment 64. The method of Embodiment 63, wherein the subject is suffering from a clinically significant adverse reaction.

Embodiment 65. The method of Embodiment 63 or 64, wherein the subject is monitored for acute hemolysis or anemia, or both, for a period of about 1 day to about 14 days, from about 1 day to about 12 days, from about 1 day to about 10 days, from about 2 days to about 14 days, from about 2 days to about 12 days, from about 2 days to about 10 days, from about 2 days to about 9 days, from about 2 days to about 5 days, from about 2 days to about 4 days, from about 5 days to about 9 days, from about 5 days to about 8 days, and from about 6 days to about 8 days.

Embodiment 66. The method of Embodiment 63 or 64, wherein the subject is monitored for about 14 days or less.

Embodiment 67. The method of Embodiments 63 or 64, wherein the subject is monitored for about 7 days or less.

Embodiment 68. The method of any one of Embodiments 1 to 67, wherein the mitapivat or pharmaceutically acceptable salt thereof is in a crystalline form.

Embodiment 69. The method of any one of Embodiments 1 to 68, wherein the mitapivat or pharmaceutically acceptable salt thereof is amorphous.

Embodiment 70. The method of any one of Embodiments 1 to 69, wherein the subject is administered mitapivat.

Embodiment 71. The method of any one of Embodiments 1 to 69, wherein the subject is administered a pharmaceutically acceptable salt of mitapivat.

Embodiment 72. The method of any one of Embodiments 1 to 69 and 71, wherein the subject is administered a hemisulfate salt of mitapivat.

Embodiment 73. The method of any one of Embodiments 1 to 69, 71, and 72, wherein the subject is administered a hemisulfate sesquihydrate salt of mitapivat.

Embodiment 74. A method of discontinuing treatment with mitapivat or a pharmaceutically acceptable thereof for a subject being administered about 5 mg of mitapivat twice daily (BID) or an amount of a pharmaceutically acceptable salt of mitapivat that is equal to about 5 mg of mitapivat BID, the method comprising administering mitapivat or a pharmaceutically acceptable thereof according to the following dose taper schedule: Day 1 to Day 7: administering about 5 mg of mitapivat once daily (QD) to the subject or administering an amount of a pharmaceutically acceptable salt of mitapivat that is equal to about 5 mg of mitapivat QD to the subject; Day 8: discontinuing the administration of mitapivat or the pharmaceutically acceptable salt of mitapivat thereafter.

Embodiment 75. A method of discontinuing treatment with mitapivat or a pharmaceutically acceptable salt thereof for a subject being administered about 5 mg of mitapivat twice daily (BID) or an amount of a pharmaceutically acceptable salt of mitapivat that is equal to about 5 mg of mitapivat BID, the method comprising administering mitapivat or a pharmaceutically acceptable thereof according to the following dose taper schedule: Day 1 to Day 7: administering about 5 mg of mitapivat once daily (QD) to the subject or administering an amount of a pharmaceutically acceptable salt of mitapivat that is equal to about 5 mg of mitapivat QD to the subject; Day 8 to Day 14: administering about 5 mg of mitapivat every other day (QOD) to the subject or administering an amount of a pharmaceutically acceptable salt of mitapivat that is equal to about 5 mg of mitapivat QOD to the subject; Day 15: discontinuing the administration of mitapivat or the pharmaceutically acceptable salt thereof.

Embodiment 76. A method of discontinuing treatment with mitapivat or a pharmaceutically acceptable salt thereof for a subject being administered about 20 mg of mitapivat twice daily (BID) or an amount of a pharmaceutically acceptable salt of mitapivat that is equal to about 20 mg of mitapivat BID, the method comprising administering mitapivat or a pharmaceutically acceptable thereof according to the following dose taper schedule: Day 1 to Day 7: administering about 20 mg of mitapivat once daily (QD) to the subject or administering an amount of a pharmaceutically acceptable salt of mitapivat that is equal to about 20 mg of mitapivat QD to the subject; Day 8 to Day 14: administering about 5 mg of mitapivat once daily (QD) to the subject or administering an amount of a pharmaceutically acceptable salt of mitapivat that is equal to about 5 mg of mitapivat QD to the subject; Day 15: discontinuing the administration of mitapivat or the pharmaceutically acceptable salt thereof.

Embodiment 77. A method of discontinuing treatment with mitapivat or a pharmaceutically acceptable salt thereof for a subject being administered about 20 mg of mitapivat twice daily (BID) or an amount of a pharmaceutically acceptable salt of mitapivat that is equal to about 20 mg of mitapivat BID to the subject, the method comprising administering mitapivat or a pharmaceutically acceptable thereof according to the following dose taper schedule: Day 1 to Day 7: administering about 20 mg of mitapivat once daily (QD) to the subject or administering an amount of a pharmaceutically acceptable salt of mitapivat that is equal to about 20 mg of mitapivat QD to the subject; Day 8 to Day 14: administering about 20 mg of mitapivat every other day (QOD) to the subject or administering an amount of a pharmaceutically acceptable salt of mitapivat that is equal to about 20 mg of mitapivat QOD to the subject; Day 15: discontinuing the administration of mitapivat or the pharmaceutically acceptable salt thereof.

Embodiment 78. A method of discontinuing treatment with mitapivat or a pharmaceutically acceptable salt thereof for a subject being administered about 50 mg of mitapivat twice daily (BID) or administering an amount of a pharmaceutically acceptable salt of mitapivat that is equal to about 50 mg of mitapivat BID to the subject, the method comprising administering mitapivat or a pharmaceutically acceptable thereof according to the following dose taper schedule: Day 1 to Day 7: administering about 50 mg of mitapivat once daily (QD) to the subject or administering an amount of a pharmaceutically acceptable salt of mitapivat that is equal to about 50 mg of mitapivat QD to the subject; Day 8 to Day 14: administering about 20 mg of mitapivat once daily (QD) to the subject or administering an amount of a pharmaceutically acceptable salt of mitapivat that is equal to about 20 mg of mitapivat QD to the subject; Day 15: discontinuing the administration of mitapivat or the pharmaceutically acceptable salt thereof.

Embodiment 79. A method of discontinuing treatment with mitapivat or a pharmaceutically acceptable salt thereof for a subject being administered about 50 mg of mitapivat twice daily (BID) or administering an amount of a pharmaceutically acceptable salt of mitapivat that is equal to about 50 mg of mitapivat BID to the subject, the method comprising administering mitapivat or a pharmaceutically acceptable thereof according to the following dose taper schedule: Day 1 to Day 7: administering about 50 mg of mitapivat once daily (QD) to the subject or administering an amount of a pharmaceutically acceptable salt of mitapivat that is equal to about 50 mg of mitapivat QD to the subject; Day 8 to Day 14: administering about 20 mg of mitapivat once daily (QD) to the subject or administering an amount of a pharmaceutically acceptable salt of mitapivat that is equal to about 20 mg of mitapivat QD to the subject; Day 15 to Day 21 administering about 20 mg of mitapivat every other day (QOD) to the subject or administering an amount of a pharmaceutically acceptable salt of mitapivat that is equal to about 20 mg of mitapivat QOD to the subject; Day 22: discontinuing the administration of mitapivat or the pharmaceutically acceptable salt thereof.

Embodiment 80. The method of any one of Embodiments 74 to 79, further comprising monitoring the subject for acute hemolysis or anemia, or both.

Embodiment 81. The method of any one of Embodiments 74 to 80, further comprising the step of: c) treating the subject for acute hemolysis or anemia, or both, if the subject shows symptoms of acute hemolysis or anemia, or both.

Embodiment 82. The method of any one of Embodiments 74 to 81, wherein the subject is being treated for a condition selected from pyruvate kinase deficiency (PKD), thalassemia, and sickle cell disease (SCD).

Embodiment 83. The method of any one of Embodiments 74 to 82, wherein the subject is being treated for hemolytic anemia or anemia caused by or associated with pyruvate kinase deficiency (PKD).

Embodiment 84. The method of any one of Embodiments 74 to 82, wherein the subject is being treated for sickle cell disease (SCD).

Embodiment 85. The method of embodiment 82, wherein the thalassemia is non-transfusion dependent thalassemia or transfusion dependent thalassemia.

Embodiment 86. The method of embodiment 82 or 85, wherein the thalassemia is alpha thalassemia or beta thalassemia.

Embodiment 87. The method of any one of Embodiments 74 to 86, wherein the mitapivat or pharmaceutically acceptable salt thereof is in a crystalline form.

Embodiment 88. The method of any one of Embodiments 74 to 86, wherein the mitapivat or pharmaceutically acceptable salt thereof is amorphous.

Embodiment 89. The method of any one of Embodiments 74 to 88, wherein the subject is being administered mitapivat prior to the dose taper schedule.

Embodiment 90. The method of any one of Embodiments 74 to 89, wherein the subject is administered mitapivat in the amounts recited by the dose taper schedule.

Embodiment 91. The method of any one of Embodiments 74 to 88, wherein the subject is being administered a pharmaceutically acceptable salt of mitapivat prior to the dose taper schedule.

Embodiment 92. The method of any one of Embodiments 74 to 88 and 91, wherein the subject is administered a pharmaceutically acceptable salt of mitapivat in the amounts recited by the dose taper schedule.

Embodiment 93. The method of any one of Embodiments 74 to 88, 91, and 92, wherein the subject is administered a hemisulfate salt of mitapivat.

Embodiment 94. The method of any one of Embodiments 74 to 88 and 91 to 93, wherein the subject is administered a hemisulfate sesquihydrate salt of mitapivat.

Embodiment 95. A kit comprising a taper pack comprising one or more oral dosage forms selected from about 5 mg, about 20 mg, and about 50 mg of mitapivat or a pharmaceutically acceptable salt thereof in an amount equal to a dosage form of about 5 mg mitapivat, about 20 mg mitapivat, and about 50 mg of mitapivat.

Embodiment 96. The kit of Embodiment 95, wherein the mitapivat or pharmaceutically acceptable salt thereof is to be administered according to any of the methods of embodiments 1-94 to minimize the risk of acute hemolysis or anemia, or both.

Embodiment 97. The kit of Embodiment 95 or 96, wherein the taper pack comprises one to about seven oral doses selected from about 5 mg, about 20 mg, and about 50 mg of mitapivat or a pharmaceutically acceptable salt thereof in an amount equal to a dose selected from about 5 mg, about 20 mg, and about 50 mg of mitapivat.

Embodiment 98. The kit of any one of Embodiments 95 to 97, wherein the taper pack comprises one to about seven oral doses of about 5 mg of mitapivat or a pharmaceutically acceptable salt thereof in an amount equal to about 5 mg of mitapivat.

Embodiment 99. The kit of any one of Embodiments 95 to 97, wherein the taper pack comprises one to about seven oral doses of about 5 mg mitapivat and one to about seven oral doses of about 20 mg of mitapivat or a pharmaceutically acceptable salt thereof in an amount equal to about 5 mg and about 20 mg of mitapivat.

Embodiment 100. The kit of any one of Embodiments 95 to 97, wherein the taper pack comprises one to about seven oral doses of about 20 mg mitapivat and one to about seven oral doses of about 50 mg of mitapivat or a pharmaceutically acceptable salt thereof in an amount equal to about 20 mg or about 50 mg of mitapivat.

While a number of embodiments have been described, the scope of this disclosure is to be defined by the appended claims, and not by the specific embodiments that have been represented by way of example. The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A method of discontinuing treatment with mitapivat or a pharmaceutically acceptable thereof for a subject being administered about 5 mg of mitapivat twice daily (BID) or an amount of a pharmaceutically acceptable salt of mitapivat that is equal to about 5 mg of mitapivat BID, the method comprising administering mitapivat or a pharmaceutically acceptable thereof according to the following dose taper schedule:
Day 1 to Day 7: administering about 5 mg of mitapivat once daily (QD) to the subject or administering an amount of a pharmaceutically acceptable salt of mitapivat that is equal to about 5 mg of mitapivat QD to the subject;
Day 8: discontinuing the administration of mitapivat or the pharmaceutically acceptable salt of mitapivat thereafter.

2. A method of discontinuing treatment with mitapivat or a pharmaceutically acceptable salt thereof for a subject being administered about 5 mg of mitapivat twice daily (BID) or an amount of a pharmaceutically acceptable salt of mitapivat that is equal to about 5 mg of mitapivat BID, the method comprising administering mitapivat or a pharmaceutically acceptable thereof according to the following dose taper schedule:
Day 1 to Day 7: administering about 5 mg of mitapivat once daily (QD) to the subject or administering an amount of a pharmaceutically acceptable salt of mitapivat that is equal to about 5 mg of mitapivat QD to the subject;
Day 8 to Day 14: administering about 5 mg of mitapivat every other day (QOD) to the subject or administering an amount of a pharmaceutically acceptable salt of mitapivat that is equal to about 5 mg of mitapivat QOD to the subject;
Day 15: discontinuing the administration of mitapivat or the pharmaceutically acceptable salt thereof.

3. A method of discontinuing treatment with mitapivat or a pharmaceutically acceptable salt thereof for a subject being administered about 20 mg of mitapivat twice daily (BID) or an amount of a pharmaceutically acceptable salt of mitapivat that is equal to about 20 mg of mitapivat BID, the method comprising administering mitapivat or a pharmaceutically acceptable thereof according to the following dose taper schedule:
Day 1 to Day 7: administering about 20 mg of mitapivat once daily (QD) to the subject or administering an amount of a pharmaceutically acceptable salt of mitapivat that is equal to about 20 mg of mitapivat QD to the subject;
Day 8 to Day 14: administering about 5 mg of mitapivat once daily (QD) to the subject or administering an amount of a pharmaceutically acceptable salt of mitapivat that is equal to about 5 mg of mitapivat QD to the subject;
Day 15: discontinuing the administration of mitapivat or the pharmaceutically acceptable salt thereof.

4. A method of discontinuing treatment with mitapivat or a pharmaceutically acceptable salt thereof for a subject being administered about 20 mg of mitapivat twice daily (BID) or an amount of a pharmaceutically acceptable salt of mitapivat that is equal to about 20 mg of mitapivat BID to the subject, the method comprising administering mitapivat or a pharmaceutically acceptable thereof according to the following dose taper schedule:
Day 1 to Day 7: administering about 20 mg of mitapivat once daily (QD) to the subject or administering an amount of a pharmaceutically acceptable salt of mitapivat that is equal to about 20 mg of mitapivat QD to the subject;
Day 8 to Day 14: administering about 20 mg of mitapivat every other day (QOD) to the subject or administering an amount of a pharmaceutically acceptable salt of mitapivat that is equal to about 20 mg of mitapivat QOD to the subject;
Day 15: discontinuing the administration of mitapivat or the pharmaceutically acceptable salt thereof.

5. A method of discontinuing treatment with mitapivat or a pharmaceutically acceptable salt thereof for a subject being administered about 50 mg of mitapivat twice daily (BID) or administering an amount of a pharmaceutically acceptable salt of mitapivat that is equal to about 50 mg of mitapivat BID to the subject, the method comprising administering mitapivat or a pharmaceutically acceptable thereof according to the following dose taper schedule:
Day 1 to Day 7: administering about 50 mg of mitapivat once daily (QD) to the subject or administering an amount of a pharmaceutically acceptable salt of mitapivat that is equal to about 50 mg of mitapivat QD to the subject;
Day 8 to Day 14: administering about 20 mg of mitapivat once daily (QD) to the subject or administering an amount of a pharmaceutically acceptable salt of mitapivat that is equal to about 20 mg of mitapivat QD to the subject;
Day 15: discontinuing the administration of mitapivat or the pharmaceutically acceptable salt thereof.

6. A method of discontinuing treatment with mitapivat or a pharmaceutically acceptable salt thereof for a subject being administered about 50 mg of mitapivat twice daily (BID) or administering an amount of a pharmaceutically acceptable salt of mitapivat that is equal to about 50 mg of mitapivat BID to the subject, the method comprising administering mitapivat or a pharmaceutically acceptable thereof according to the following dose taper schedule:
Day 1 to Day 7: administering about 50 mg of mitapivat once daily (QD) to the subject or administering an amount of a pharmaceutically acceptable salt of mitapivat that is equal to about 50 mg of mitapivat QD to the subject;
Day 8 to Day 14: administering about 20 mg of mitapivat once daily (QD) to the subject or administering an amount of a pharmaceutically acceptable salt of mitapivat that is equal to about 20 mg of mitapivat QD to the subject;
Day 15 to Day 21 administering about 20 mg of mitapivat every other day (QOD) to the subject or administering an amount of a pharmaceutically acceptable salt of mitapivat that is equal to about 20 mg of mitapivat QOD to the subject;
Day 22: discontinuing the administration of mitapivat or the pharmaceutically acceptable salt thereof.

7. The method of any one of claims 1 to 6, further comprising monitoring the subject for acute hemolysis or anemia, or both.

8. The method of any one of claims 1 to 7, further comprising the step of: c) treating the subject for acute hemolysis or anemia, or both, if the subject shows symptoms of acute hemolysis or anemia, or both.

9. The method of any one of claims 1 to 8, wherein the subject is being treated for a condition selected from pyruvate kinase deficiency (PKD), thalassemia, and sickle cell disease (SCD).

10. The method of any one of claims 1 to 9, wherein the subject is being treated for hemolytic anemia or anemia caused by or associated with pyruvate kinase deficiency (PKD).

11. The method of any one of claims 1 to 9, wherein the subject is being treated for sickle cell disease (SCD).

12. The method of any one of claims 1 to 9, wherein the subject is being treated for thalassemia.

13. The method of claim 12, wherein the thalassemia is non-transfusion dependent thalassemia or transfusion dependent thalassemia.

14. The method of claim 12 or 13, wherein the thalassemia is alpha thalassemia or beta thalassemia.

15. The method of any one of claims 1 to 14, wherein the mitapivat or pharmaceutically acceptable salt thereof is in a crystalline form.

16. The method of any one of claims 1 to 14, wherein the mitapivat or pharmaceutically acceptable salt thereof is amorphous.

17. The method of any one of claims 1 to 16, wherein the subject is being administered mitapivat prior to the dose taper schedule.

18. The method of any one of claims 1 to 17, wherein the subject is administered mitapivat in the amounts recited by the dose taper schedule.

19. The method of any one of claims 1 to 16, wherein the subject is being administered a pharmaceutically acceptable salt of mitapivat prior to the dose taper schedule.

20. The method of any one of claims 1 to 16 and 19, wherein the subject is administered a pharmaceutically acceptable salt of mitapivat in the amounts recited by the dose taper schedule.

21. The method of any one of claims 1 to 16, 19, and 20, wherein the subject is administered a hemisulfate salt of mitapivat.

22. The method of any one of claims 1 to 16 and 19 to 21, wherein the subject is administered a hemisulfate sesquihydrate salt of mitapivat.

23. A kit comprising a taper pack comprising one or more oral dosage forms selected from about 5 mg, about 20 mg, and about 50 mg of mitapivat or a pharmaceutically acceptable salt thereof in an amount equal to a dosage form of about 5 mg mitapivat, about 20 mg mitapivat, and about 50 mg of mitapivat.

24. The kit of claim 23, wherein the mitapivat or pharmaceutically acceptable salt thereof is to be administered according to any of the methods of claims 1-22 to minimize the risk of acute hemolysis or anemia, or both.

25. The kit of claim 23 or 24, wherein the taper pack comprises one to about seven oral doses selected from about 5 mg, about 20 mg, and about 50 mg of mitapivat or a pharmaceutically acceptable salt thereof in an amount equal to a dose selected from about 5 mg, about 20 mg, and about 50 mg of mitapivat.

26. The kit of any one of claims 23 to 25, wherein the taper pack comprises one to about seven oral doses of about 5 mg of mitapivat or a pharmaceutically acceptable salt thereof in an amount equal to about 5 mg of mitapivat.

27. The kit of any one of claims 23 to 26, wherein the taper pack comprises one to about seven oral doses of about 5 mg mitapivat and one to about seven oral doses of about 20 mg of mitapivat or a pharmaceutically acceptable salt thereof in an amount equal to about 5 mg and about 20 mg of mitapivat.

28. The kit of any one of claims 23 to 27, wherein the taper pack comprises one to about seven oral doses of about 20 mg mitapivat and one to about seven oral doses of about 50 mg of mitapivat or a pharmaceutically acceptable salt thereof in an amount equal to about 20 mg or about 50 mg of mitapivat.
